(19) United States
(12) Patent Application Publication
(10) Pub. No.: US 2026/0364092 A1
(43) Pub. Date: Nov. 19, 2026

(54) NOVEL USE OF AN ILEAL BILE ACID TRANSPORTER INHIBITOR FOR THE TREATMENT OF PRURITUS

(71) Applicant: GLAXOSMITHKLINE INTELLECTUAL PROPERTY (NO.2) LIMITED, Stevenage (GB)

(72) Inventors: Megan M. MCLAUGHLIN, Collegeville, PA (US); Stuart F. KENDRICK, Stevenage (GB); Usha GUNGABISSOON, Londom (GB)

(73) Assignee: GLAXOSMITHKLINE INTELLECTUAL PROPERTY (NO.2) LIMITED, Stevenage (GB)

(21) Appl. No.: 19/487,572

(22) PCT Filed: Jun. 4, 2024
(86) PCT No.: PCT/EP2024/065346

§ 371 Date: Nov. 25, 2025

Related U.S. Application Data
(60) Provisional application No. 63/506,433, filed on Jun. 6, 2023.

(51) Int. Cl. A61K 31/554; A61K 31/4995; A61K 31/7042; A61K 38/05; A61P 1/16; A61P 17/04

(52) U.S. Cl. CPC A61K 31/554; A61K 31/4995; A61K 31/7042; A61K 38/05; A61P 1/16; A61P 17/04

(57) ABSTRACT

The present invention relates to an IBAT inhibitor for use in the treatment of pruritus in certain chronic liver diseases such as non-alcoholic fatty liver disease (NAFLD) [newly reclassified as metabolic dysfunction-associated steatotic liver disease (MASLD)] which encompasses non-alcoholic fatty liver (NAFL) and non-alcoholic steatohepatitis (NASH) [newly reclassified as metabolic dysfunction-associated steatohepatitis (MASH)] with or without cirrhosis, and methods of treatment of pruritus in NAFLD (or MASLD), including NAFL and NASH (or MASH) with or without cirrhosis, comprising administering to said human a therapeutically effective amount of an IBAT inhibitor such as linerixibat.

### FIELD OF THE INVENTION

The present invention relates to an IBAT inhibitor for use in the treatment of pruritus in certain chronic liver diseases such as non-alcoholic fatty liver disease (NAFLD) [newly reclassified as metabolic dysfunction-associated steatotic liver disease (MASLD)] which encompasses non-alcoholic fatty liver (NAFL) and non-alcoholic steatohepatitis (NASH) [newly reclassified as metabolic dysfunction-associated steatohepatitis (MASH)] with or without cirrhosis, and methods of treatment of pruritus in NAFLD (or MASLD), including NAFL and NASH (or MASH) with or without cirrhosis, comprising administering to said human a therapeutically effective amount of an IBAT inhibitor, for example linerixibat.

### BACKGROUND TO THE INVENTION

Pruritus (itch) is a well-recognised and characterised symptom of chronic, cholestatic liver diseases such as primary biliary cholangitis (PBC) and primary sclerosing cholangitis (PSC) and is known to impact quality of life (QoL). The Primary Biliary Cholangitis: 2018 Practice Guidance from the American Association for the Study of Liver Diseases [Lindor et al., 2019 Hepatology, 69(1), 394-419] states that, “The origin of pruritus in PBC is still unknown. However, several important mediators in the pathophysiology of cholestatic pruritus, which provide opportunities for therapeutic intervention, have been identified, including lysophosphatidic acid, endogenous opioids, and bile acids.”

Cholestatic pruritus is a common often debilitating condition that significantly impairs day-to-day functioning, including sleep latency and disturbance, fatigue, depression, and suicidal ideation (Younossi, Kiwi et al. 2000; Montagnese, Nsemi et al. 2013; Hegade, Bolier et al. 2016; Jin and Khan 2016; Hönig, Herder et al. 2018, Hegade, Mells et al 2019, Mayo M J, Dig Dis Sci. 2023 March; 68(3):995-1005).

The characteristics of itch in patients with known cholestatic liver diseases in, for example PBC, has been characterized as ‘relentless’ or so severe the patients wanted to ‘tear (their) skin off’ or scratched until they bled (Rishe, Azarm et al. 2008). Further, factors that worsen itch in cholestatic liver disease include heat, psychological stress, wearing specific clothing as well as diurnal changes; it is often worse at night (Bergasa, Mehlman et al. 2000, Kremer, Beuers et al. 2008, Kremer, Oude Elferink et al. 2011, Lindor, Bowlus et al. 2019, Vander Does, Levy et al. 2022).

Drug development in liver disease historically has mainly focused on treatment of the underlying liver disease and not the symptoms, such as pruritus. Interestingly, some approved and investigational treatments for chronic liver diseases may actually result in pruritus as a side-effect or increase existing pruritus instead of alleviating it. Obeticholic acid (OCA) (OCALIVA), approved for treatment of PBC in patients with inadequate response or intolerant to ursodeoxycholic acid (UDCA), is associated with a higher incidence and severity of pruritus in PBC patients as compared to placebo (Nevins, et al., N Engl. J Med 2016; 375:631-643) and has a warning and precaution for severe pruritus in the label: [https://www.accessdata.fda.gov/drugsatfda_docs/label/2022/207999s008lbl.pdf].

As used herein:
‘NAFLD’ refers to non-alcoholic fatty liver disease, and until very recently has been used to describe the histological spectrum of steatosis to steatohepatitis with its subtypes ‘NAFL’ (non-alcoholic fatty liver (simple steatosis)) and ‘NASH’ (Non-alcoholic steatohepatitis).
‘MASLD’ refers to metabolic dysfunction associated steatotic liver disease, and represents the the new classification of the ‘NAFLD’ disease [Hepatology 78(6) 1966-1986], that is, the MASLD disease was formerly largely classified using the term NAFLD.
‘MASH’ refers to metabolic dysfunction-associated steatohepatitis and is the ‘replacement term’ for NASH (M. E. Rinella, J. V. Lazarus, V. Ratziu et al. Annals of Hepatology 29 (2024) 101133).
The investigational products obeticholic acid and tropifexor have both been found to result in increased pruritus in NASH patients [Neuschwander, et al., Lancet 2015; 385:956-65; Patel K, et al., Hepatology 2020; 72:58-71.]

Recent studies have assessed the potential of ileal bile acid transporter (IBAT) inhibitors, also known as apical sodium-dependent bile acid transporter (ASBT) inhibitors, for the treatment of pruritus in some pediatric cholestatic liver diseases. An excess of systemic bile acids in cholestasis is hypothesized to have a role in cholestatic pruritus, and the impact of lowering bile acids has supported this theory (Hepatology. 2019; 69(1):394-419; Management of cholestatic liver diseases. J Hepatol. 2009; 51(2):237-267). IBAT inhibitors block the enterohepatic circulation of bile acids, thereby reducing circulating bile acid levels and increasing fecal bile acid excretion (Lancet. 2017; 389(10074):1114-1123). The molecular IBAT inhibitors maralixibat (LIVMARLI) and odevixibat (BYLVAY) were recently approved by the FDA for the treatment of cholestatic pruritus in Alagille syndrome and pediatric familial intrahepatic cholestasis (PFIC), respectively.
[LIVMARLI https://www.accessdata.fda.gov/drugsatfda_docs/label/2021/214662s000lbl.pdf]
[BYLVAY https://www.accessdata.fda.gov/drugsatfda_docs/label/2021/215498s000lbl.pdf]. Odevixibat was also approved by the European Medicines Agency [https://www.ema.europa.eu/en/documents/product-information/bylvay-epar-product-information_en.pdf]

Linerixibat, a minimally absorbed oral small molecule IBAT inhibitor, showed significant improvements in pruritus and also reduction of serum concentration of bile acids and autotaxin (the enzyme that produces lysophosphatidic acid) versus placebo in a phase 2a and phase 2b studies of patients with PBC (Hegade, Kendrick et al. 2017) (Levy 2022 Clin Gastroenterol Hepatol. 2022, available online https://doi.org/10.1016/j.cgh.2022.10.032). The phase 2b GLIMMER study (NCT02966834) is the largest randomized investigational study of PBC patients with cholestatic pruritus to date. In this study, linerixibat dose-dependently ameliorated pruritus over 12 weeks of treatment (Levy 2022 et al.). Further, it has now been shown from our own studies (Example 5 herein) that linerixibat treatment of PBC patients with pruritus results in a decrease in total serum bile acids which correlates with itch reduction in patients with cholestatic pruritus [Karatza et al., EASL abstract: Karatza E, et al. J Hepatol 2023:78 (S1):S367; poster: Karatza et al., EASL poster 2023 https://www.postersessiononline.eu/173580348_eu/congresos/ILC2023/aula/-TOP_62_ILC2023.pdf; manuscript: https://doi.org/10.1111/liv.15982]. Together, these findings indicate the potential of linerixibat as a future treatment option for cholestatic pruritus in PBC.

However, pruritus in other chronic liver diseases (CLDs) that are not classically considered to be cholestatic is not well-recognised; this is emphasised by the limited literature in this context. There is for example, limited and less consistent data describing the presence and prevalence of pruritus in people with other CLDs such as chronic viral hepatitis (including viral hepatitis B and viral hepatitis C), autoimmune hepatitis (AIH) or NAFLD (or MASLD) and pruritus is often overlooked during the treatment of liver disease (Biernacka, Nizyński et al. 2018). Where pruritus is reported to be present in these other CLDs, there is no consensus regarding the mechanisms regulating the etiopathogenesis of pruritus. The cause of the pruritus in these other CLDs such as viral hepatitis B (HepB), viral hepatitis C (HepC), AIH and NAFLD (or MASLD), particularly in NAFLD (or MASLD), for example NASH (or MASH), is therefore poorly understood.

There are to date no publications investigating the prospective natural history of pruritus over time and its impact on patients, and limited understanding of the underlying mechanism of pruritus in this population of patients with CLDs not classically considered to be cholestatic, namely in patients with HepB, HepC, AIH and NAFLD (or MASLD), particularly in patients with NAFLD (or MASLD), for example NASH (or MASH).

Furthermore, in chronic liver diseases such as NAFLD (or MASLD) and its progressive form NASH (or MASH), (Biomedicines 2022, 10, 451; Hepatology Communications, VOL. 4, NO. 11, 2020) and in cholestatic liver diseases such as PBC and PSC, patients experience pruritus with significant impairment in quality of life (Frontiers in Medicine 2021 Volume 8: Article 639674; Proteome Res. 2021, 20, 2340-2351; European Journal of Gastroenterology & Hepatology: December 2014-Volume 26-Issue 12-p 1374-1379).

An effective treatment for pruritus in HepB, HepC, AIH and NAFLD (or MASLD), for example NASH (or MASH), is expected to significantly improve the quality of life in those patients with a pruritus component in such CLDs. Thus, an effective treatment for pruritus in HepB, HepC, AIH and NAFLD (or MASLD), for example NASH (or MASH), is highly desirable.

### SUMMARY OF THE INVENTION

In a first aspect of the invention there is provided an IBAT inhibitor for use in the treatment of pruritus in NAFLD (or MASLD).

In a second aspect of the invention there is provided an IBAT inhibitor for use in the treatment of cholestatic pruritus in NAFLD (or MASLD).

In a third aspect of the invention there is provided a method of treatment of pruritus in NAFLD (or MASLD) in a human in need thereof, comprising administering to said human a therapeutically effective amount of an IBAT inhibitor.

In fourth aspect of the invention there is provided a method of treatment of cholestatic pruritus in NAFLD (or MASLD) in a human in need thereof, comprising administering to said human a therapeutically effective amount of an IBAT inhibitor.

### DESCRIPTION OF DRAWINGS/FIGURES

FIG. 1 is a 5D-Itch Plot of Natural History Study Results for NASH (or MASH), PBC, PSC and AIH vs Non-Cholestatic Prior Art Pruritus Conditions, from Example 2a

FIG. 1a is the same 5D-Itch Plot of Natural History Study Results as FIG. 1. showing data for NASH (or MASH) and PBC only

shows the same 5D-Itch Plot of Natural History Study Results, for NASH (or MASH) and PBC only

FIG. 2 is a Sankey diagram showing change in itch severity over time in NASH (or MASH) and PBC, from Example 1

FIG. 3 is a plot of individual Bayesian estimate of TSBA change from baseline AUC₀₋₂₄ with change in weekly itch score over a 12-week treatment period of PBC patients, from Example 5

FIG. 4A is a plot of ATX Least Squares mean ratio to baseline at Week 16 in itch responders versus non-responders treated with 40/90 mg BID linerixibat or placebo, of PBC patients from Example 6

FIG. 4B is a plot of ATX Least Squares mean ratio of linerixibat versus placebo treatment in itch responders and non-responders, of PBC patients from Example 6

### DETAILED DESCRIPTION OF THE INVENTION

### Measurement of Pruritus, Including Cholestatic Pruritus

Pruritus can be measured using different rating scales such as:
i) 0-10-point Numerical Rating Scale (NRS): The patient-reported NRS scale measures intensity or severity of itch (rather than frequency of itch). For example, the use of NRS in the measurement of pruritus in PBC: “Development and adaptation of patient-reported outcome measures for patients who experience itch associated with primary biliary cholangitis.” J Patient Rep Outcomes. 2019; 3(1):2.
ii) 5D Itch Scale: Elman et al., “The 5-D itch scale: a new measure of pruritus.” Br J Dermatol. 2010 March; 162(3):587-93. The 5-D itch scale is a multidimensional patient-reported questionnaire designed to measure the following five dimensions: degree, duration, direction, disability and distribution.
iii) For patients with PBC: PBC-40 (PBC-40): Jacoby et al., “Development, validation, and evaluation of the PBC-40, a disease specific health related quality of life measure for primary biliary cirrhosis.” Gut. 2005 November; 54(11):1622-9. The six domains of PBC-40, a patient-reported questionnaire, relate to fatigue, emotional, social, and cognitive function, general symptoms, and itch.

There are no well-established physical or physiological markers of pruritus severity or treatment efficacy associated with cholestatic liver disease that can be observed or measured. The rating of itch is subjective, and each participant is likely to have a distinct response to any treatment for cholestatic pruritus. Therefore, patient-reported outcomes (PRO) are the best way to assess itch severity and measure a treatment effect. Treatment effect may be measured by examining the change of itch from baseline when administering a treatment and comparing it with change of itch from baseline when administering a placebo. Alternatively, one can compare the number of patients classified as a responder on a given treatment, with the number on placebo, wherein a responder is defined as a participant that improves by, or more than, the predefined responder.

The pruritus component in patients suffering from a CLD such as HepB, HepC, AIH and NAFLD (or MASLD), for example NASH (or MASH), can be classed as mild, moderate or severe, using various different measurement tools and thresholds such as Numerical Rating Scale (NRS), Visual Analogue Scale (VAS), Verbal Rating Scale (VRS). The pruritus can result in a poor quality of life for patients, particularly for those suffering from moderate to severe pruritus.

Below is a summary of the current understanding in the art of the chronic liver diseases HepB, HepC, AIH and NAFLD (or MASLD), including NASH (or MASH), and the extent to which pruritus has been reported in the art in patients with these diseases.

### Non-Alcoholic Fatty Liver Disease (NAFLD); Non-Alcoholic Steatohepatitis (NASH); Metabolic Dysfunction Associated Steatotic Liver Disease (MASLD); Metabolic Dysfunction Associated Steatohepatitis (MASH)

Non-alcoholic fatty liver disease (NAFLD) (newly classified as metabolic dysfunction associated steatotic liver disease (MASLD)) is the fastest growing cause of chronic liver disease and the leading cause of cirrhosis and hepatocellular carcinoma and a growing indication for liver transplantation worldwide. MASLD is the new term employed to refer to steatotic liver disease related to systemic metabolic dysregulation, and encompasses patients who have hepatic steatosis and have at least one of five cardiometabolic risk factors. The term NAFLD was until very recently used to describe the histological spectrum of steatosis to steatohepatitis with its subtypes NAFL and NASH. NAFLD encompasses a disease continuum and exists in two clinical entities; non-alcoholic fatty liver (NAFL, simple steatosis) and non-alcoholic steatohepatitis (NASH), with or without cirrhosis. Classically, NAFL is considered a relatively benign disease which does not usually progress to NASH and liver fibrosis. However, recent clinical data from paired liver-biopsy studies have shown that around 25% of patients with NAFL progressed to NASH and bridging fibrosis (Pais R and Maurel T. J. Clin. Med. 2021, 10, 1161). NASH, on the other hand, is characterised by necroinflammation and faster fibrosis progression than simple steatosis, i.e. NAFL (Fiorucci, Biagioli et al. 2018, Powell, Wong et al. 2021). NASH (or MASH) is considered a progressive liver disease with a significant risk for development of cirrhosis and liver-related morbidities and mortality. Furthermore, in late stages of certain chronic liver diseases including NASH, cholestatic features may be observed (Eur J Clin Invest 2013; 43 (10): 1069-1083). Currently, it is estimated that 25% of the world population have NAFLD (or MASLD), 20%-30% of patients with NAFLD (or MASLD) will have NASH (or MASH) and that 10%-15% of these can progress to cirrhosis (Armandi A and Bugianesi E. Liver International. 2021; 41(Suppl. 1):78-82).

Estimates from cross-sectional studies in the published literature report that between 14 and 55% of patients with NAFLD (or MASLD) or NASH (or MASH) experience pruritus at any given point in time. If restricted to those with moderate to severe itch, the prevalence at any given point ranges from 20 to 27% of patients. The literature typically describes itch in the broader NAFLD (or MASLD) population rather than specifically restricting to the subset with NASH (or MASH).

To the best of our knowledge, there are no global longitudinal evaluations of pruritus and its impact over time in the general NAFLD (or MASLD) or NASH (or MASH) populations, in the prior art.

There is little to no documentation about the cause of pruritus in NASH (or MASH) (Vander Does, A., Levy, C. & Yosipovitch, G. Cholestatic Itch: Our Current Understanding of Pathophysiology and Treatments. Am J Clin Dermatol 23, 647-659 (2022)).

There is a lack of treatments for the unmet need of pruritus in NASH (or MASH). There are no official guidelines or approved therapies for the treatment of pruritus in NASH (or MASH) in the United States or European Union. Based on the totality of the evidence from the literature and our own studies, particularly the Examples described herein, there is an unmet medical need for a treatment for pruritus in NASH (or MASH).

TABLE A
Pruritus prevalence in NASH (or MASH) or NAFLD (or MASLD) reported in the literature

| | | | | Time | | Prevalence (%) | Ascertainment |
|---|---|---|---|---|---|---|---|
| Citation | Population | N | Country | period | Setting | of pruritus | of pruritus |
| (Fujino, | NAFLD | 289 | Japan | 2016- | Patients attending Hiroshima | ~30% any | NRS |
| Tanaka | | | | 2017 | University Hospital | severity | |
| et al. 2019) | | | | | | ~20% | |
| | | | | | | moderate- | |
| | | | | | | severe | |
| (Kido- | NAFLD | 14 | Japan | 2015- | Patients attending | 14.3% any | VAS |
| Nakahara, | | | | 2016 | department of internal | severity | |
| Nakahara | | | | | medicine at one of three | | |
| et al. 2019) | | | | | general hospitals | | |
| (Oeda, | NAFLD | 338 | Japan | 2016 | Outpatients from 9 facilities | 44.7% any | VAS |
| Takahashi | | | | | | severity | |
| et al. 2018) | | | | | | | |
| (Boehlig, | NAFLD | 123 | Germany | 2019- | Outpatients at a tertiary | 41.8% any | 5D Itch |
| Gerhardt | | | | 2020 | referral centre | severity | |
| et al. 2022) | | | | | | 20.9% mild | |
| | | | | | | 18.2% | |
| | | | | | | moderate | |
| | | | | | | 2.7% severe | |
| (Whalley, | NASH - | 152 | US | NS | Placebo-controlled trial of | 55% any | NASH-CHECK |
| Twiss | stage 2 or | | | | tropifexor - itch at baseline | severity | |
| et al. 2020) | 3 fibrotic | | | | | | |
| | (F2, F3) | | | | | | |
| (Younossi, | NASH - | 1169 | NS | NS | STELLAR phase 3 clinical | 27% clinically | CLDQ-NASH |
| Anstee | advanced | | | | trial of selonsertib. Itch at | significant itch | |
| et al. 2020) | | | | | baseline | | |
| (Foster, Baki | NASH or | 305 | US | 2018- | Prospectively enrolled | 38.7% any | 6-month |
| et al. 2020) | ALD with | | | 2019 | patients from the University | severity | patient recall |
| | cirrhosis | | | | of Michigan outpatient | | (yes/no) |
| | | | | | Hepatology clinic and | | |
| | | | | | inpatient Hepatology ward. | | |

ALD: Alcoholic liver disease
NRS: numerical rating scale - 0-10 scale ranges from no itch (0) to worst imaginable itch (10). Itch of any severity defined as NRS ≥1, moderate-severe itch as NRS ≥4
VAS: Visual analogue scale - 10-cm-long horizontal line ranges from “no itch” (0 cm) to “worst imaginable itch” (10 cm)
NASH-CHECK: A NASH disease-specific patient-reported outcome (PRO) measure which includes 0-10 itch scale, similar to the NRS.
5D itch scale: ranges from 5-25. No itch (5-8), mild (9-11), moderate (12-17), severe (18-25).
CLDQ-NASH: Chronic Liver Disease Questionnaire-NASH -- a disease-specific PRO measure which includes a single itch item with response options ranging from 1 (“all of the time”) to 7 (“none of the time”); clinically significant itch is defined as a score of ≤4

### Viral Hepatitis B

Hepatitis B is an infection of the liver which is caused by the hepatitis B virus (HBV) which can result in acute or chronic infection. Acute liver damage is caused mainly by the protective immune response, which destroys virus-infected liver cells. In the absence of an immune response sufficient to clear the virus, the infection becomes chronic (Fattovich 2003). Chronic HBV infection is defined as persistence of hepatitis B surface antigen (HBsAg) for 6 months or more after acute infection HBV. Chronic HBV infection is associated with an increased risk of liver fibrosis, cirrhosis and hepatocellular carcinoma (HCC) and associated mortality risks (NICE 2013, Tang, Covert et al. 2018). In late stages of certain chronic liver diseases including HepB, cholestatic features may be observed (Eur J Clin Invest 2013; 43 (10): 1069-1083).

The main goal of therapy in HepB is to improve survival and quality of life by preventing disease progression, and consequently development of HCC. The long-term administration of a nucleoside analogue, e.g., entecavir, tenofovir disoproxil or tenofovir alafenamide, represents the treatment of choice. Pegylated interferon-alfa treatment can also be considered in mild to moderate chronic hepatitis B patients (European Association for the Study of the Liver. Electronic address and European Association for the Study of the 2017, Terrault, Lok et al. 2018).

Estimates from cross-sectional studies from the published literature indicate that between 20 and 32% of patients with chronic HBV experience pruritus at any given timepoint; this is limited to between 8 and 12% if restricted to those reporting moderate-severe itch (NRS≥4) or itch that is bothersome.

TABLE B
Pruritus prevalence in Chronic Viral Hepatitis B reported in the literature

| | | | | Time | | Prevalence (%) | Ascertainment |
|---|---|---|---|---|---|---|---|
| Citation | Population | N | Country | period | Setting | of pruritus | of pruritus |
| (Fujino, | Chronic | 718 | Japan | 2016- | Patients attending | 25.5% any | NRS |
| Tanaka | HBV | | | 2017 | Hiroshima | severity | |
| et al. 2019) | | | | | University Hospital | ~12% - | |
| | | | | | | moderate- | |
| | | | | | | severe | |
| (Kido- | Chronic | 36 | Japan | 2015- | Patients visiting | 24.3% any | VAS |
| Nakahara, | HBV | | | 2016 | departments of | severity | |
| Nakahara | | | | | internal medicine at | | |
| et al. 2019) | | | | | 3 general hospitals | | |
| (Oeda, | Active | 175 | Japan | 2016 | Outpatients from 9 | 40.6% any | VAS |
| Takahashi | HBV or | active | | | facilities | severity (active | |
| et al. 2018) | HBV | HBV, | | | | HBV) | |
| | carriers | 54 HBV | | | | 22.2% any | |
| | | carriers | | | | severity (HBV | |
| | | | | | | carriers) | |
| (Evon, Lin | Chronic | 876 | USA | | Hepatitis B | 32% any | 10-item |
| et al. 2020) | HBV | | Canada | | Research Network | severity | Symptom |
| | | | | | Adult Cohort Study | 8% moderate- | checklist |
| | | | | | | extremely | |
| | | | | | | bothersome | |
| (Biernacka, | Chronic | 110 | Poland | not | Screening of | 20% any | Custom |
| Nizyński | HCV | | | specified | persons infected | severity | questionnaire, |
| et al. 2018) | and/or | | | | with HBV, HCV or | 8.2% | VAS and VRS |
| | HBV | | | | both | moderate- | |
| | | | | | | severe | |

NRS: numerical rating scale - 0-10 scale ranges from no itch (0) to worst imaginable itch (10). Itch of any severity defined as NRS ≥1, moderate-severe itch as NRS ≥4
VAS: Visual analogue scale - 10-cm-long horizontal line ranges from “no itch” (0 cm) to “worst imaginable itch” (10 cm)
VRS: Verbal rating scale consists of a list of phrases that describe increasing levels of pruritus intensity: no pruritus, mild pruritus, moderate pruritus, severe pruritus, and very severe pruritus.
10-Item symptom checklist: Participant indicates how bothered they were by the symptom in the last month: None at all (0), A little bit (1), Moderately (2), Quite a bit (3) or Extremely (4).

### Viral Hepatitis C

Chronic hepatitis C virus (HCV) infection can lead to progressive liver disease with the development of liver cirrhosis and HCC, possibly accounting for up to 0.5 million deaths every year (Wedemeyer, Dore et al. 2015). Around 30% of infected persons spontaneously clear the virus within 6 months of infection without any treatment. The remaining 70% will develop chronic HCV infection. Globally, an estimated 71 million people have chronic hepatitis C virus infection (WHO 2019). In late stages of certain chronic liver diseases including HepC, cholestatic features may be observed (Eur J Clin Invest 2013; 43 (10): 1069-1083).

Six different genotypes (HCV-1 to HCV-6) and several subtypes have been identified, with different geographical and virulence patterns and different response to conventional therapy. Genotype 1 (subtype 1a and 1b) is by far the most prevalent genotype worldwide. It is mainly transmitted by the parenteral route, although it may be also transmitted by sexual intercourse and by the mother-to-child route (Zaltron, Spinetti et al. 2012).

Clinical care for patients with HCV-related liver disease has advanced considerably during the last couple of decades. The primary goal of HCV therapy is to cure the infection, i.e. to achieve a sustained virological response (SVR) defined as undetectable HCV RNA after treatment completion (Journal of Hepatology 2020 vol. 73, 1170-1218). The WHO recommends the use of direct-acting antiviral (DAA) regimens for the treatment of all persons with hepatitis C infection (Guidelines for the care and treatment of persons diagnosed with chronic hepatitis C virus infection, World Health Organization 2018). Overall, DAA regimens successfully cure HCV infection in >95% of treated persons (Ann Intern Med 2017; 166:637-648) (Hepatology, VOL. 71, NO. 2, 2020).

Pruritus is reported to affect between 15 and 58% of patients with chronic HCV or between 8 and 42% if restricted to moderate-severe itch based on cross-sectional studies reported in the published literature, below.

TABLE C
Pruritus prevalence in Viral Hepatitis C reported in the literature

| | Population | | | Time | | Prevalence (%) | Ascertainment |
|---|---|---|---|---|---|---|---|
| Citation | etc) | N | Country | period | Setting | of pruritus | of pruritus |
| (Biernacka, | Chronic | 110 | Poland | not | Screening of | 20% any severity | Custom |
| Nizyński | HCV | | | specified | persons infected | 8.2% moderate- | questionnaire, |
| et al. 2018) | and/or | | | | with HBV, HCV or | severe | VAS and VRS |
| | HBV | | | | both | | |
| (Cacoub, | Chronic | 1,614 | France | <=1997 | Single center cohort | 15% any severity | Questionnaire- |
| Poynard | HCV | | | | (prior to HCV | | based |
| et al. 1999) | | | | | treatment initiation) | | (instrument |
| | | | | | | | not specified) |
| (Fujino, | Chronic | 1114 | Japan | 2016- | Patients with | 29.7% any | NRS |
| Tanaka | HCV | | | 2017 | chronic liver disease | severity | |
| et al. 2019) | | | | | attending Hiroshima | ~15% moderate- | |
| | | | | | University Hospital | severe | |
| (Kido- | Chronic | 116 | Japan | 2015- | patients who visited | 44.4% any | VAS and VRS |
| Nakahara, | HCV | | | 2016 | departments of | severity | |
| Nakahara | | | | | internal medicine at | | |
| et al. 2019) | | | | | 3 general hospitals | | |
| (Oeda, | Chronic | 366 | Japan | January- | outpatients from 9 | 45.9% any | VAS |
| Takahashi | HCV | | | June 2016 | facilities | severity (chronic | |
| et al. 2018) | | | | | | HCV) | |
| | | | | | | 45.9% any | |
| | | | | | | severity (post | |
| | | | | | | sustained virologic | |
| | | | | | | response svr) | |
| (Suzuki, | Chronic | 89 | Japan | October | | 58.4% any | VAS for |
| Tamano | HCV | | | 2010- | | severity (nocturnal | diurnal |
| et al. 2014) | | | | September | | pruritus) | pruritus & 5- |
| | | | | 2012 | | 41.6% moderate- | point scale |
| | | | | | | severe (nocturnal | for nocturnal |
| | | | | | | pruritus) | pruritus |

NRS: numerical rating scale - 0-10 scale ranges from no itch (0) to worst imaginable itch (10). Itch of any severity defined as NRS ≥1, moderate-severe itch as NRS ≥4
VAS: Visual analogue scale - 10-cm-long horizontal line ranges from “no itch” (0 cm) to “worst imaginable itch” (10 cm)
VRS: Verbal rating scale consists of a list of phrases that describe increasing levels of pruritus intensity: no pruritus, mild pruritus, moderate pruritus, severe pruritus, and very severe pruritus.

### Autoimmune Hepatitis (AIH)

Autoimmune hepatitis (AIH) is a chronic autoimmune inflammatory liver disease that occurs globally; it affects all ages and has a female predominance. AIH is a rare disease and is estimated to affect around 20 people per 100,000 population in Europe (Mieli-Vergani, Vergani et al. 2018). Although AIH by definition is a chronic disease that may lead to cirrhosis, HCC, liver transplantation and/or death, it can present as fulminant hepatic failure and therefore must be considered in the differential diagnosis of acute liver failure (Manns, Lohse et al. 2015). The diagnosis of AIH is based on histological abnormalities (interface hepatitis), characteristic clinical and laboratory findings (elevated serum aspartate aminotransferase [AST] and alanine aminotransferase [ALT] levels and increased serum IgG concentration), and the presence of one or more characteristic autoantibodies. (Liberal, Krawitt et al. 2016; Hepatology, Vol. 72, No. 2, 2020).

AIH can have cholestatic features that are outside the codified diagnostic criteria. These features have uncertain effects on the clinical presentation and progression of disease. Patients with AIH can have antimitochondrial antibodies and coincidental bile duct injury or loss (2%-13% of patients), focal biliary strictures and dilations based on cholangiography (2%-11%), or histologic changes of bile duct injury or loss in the absence of other features (5%-11%).

AIH is classified into two types: AIH type 1 (AIH-1), positive for anti-nuclear antibodies (ANA) and/or anti-smooth muscle antibodies (SMA), and AIH type 2 (AIH-2), positive for anti-liver kidney microsomal antibody type 1 (anti-LKM1), anti-LKM3 and/or anti-liver cytosol type 1 antibody (anti-LC1) (Manns, Lohse et al. 2015). AIH-1 affects people of all ages with two peaks, one between 10 years and 18 years of age and the other in adulthood around the age of 40 years. AIH-2 mainly affects children, including infants, adolescents and young adults defined as less than 25 years of age (Muratori, Fabbri et al. 2015, Mieli-Vergani, Vergani et al. 2018).

AIH is asymptomatic in 25%-34% of patients. Easy fatigability is the main complaint in 85% of patients, and jaundice may be present. The objectives of first-line therapy are to improve symptoms, control hepatic inflammation, achieve biochemical remission, prevent disease progression, and promote the regression of fibrosis at the lowest risk of drug-induced complication. The ideal laboratory response is normalization of serum ALT, AST, and IgG levels. Medication to induce remission consists of either high dose corticosteroids alone or in combination with azathioprine. Histological features of NAFLD (or MASLD) are present in 17%-30% of adult patients with AIH, and concurrent NAFLD (or MASLD) may influence response to therapy (Hepatology, Vol. 72, No. 2, 2020).

Pruritus is reported to affect between 4 and 55% of patients with AIH or between 18 and 42% if restricted to moderate-severe or significant itch.

TABLE D
Pruritus prevalence in AIH reported in the literature

| | | | | Time | | Prevalence (%) | Pruritus |
|---|---|---|---|---|---|---|---|
| Citation | Population | N | Country | period | Setting | of pruritus | definition |
| (Abdollahi | AIH | 60 | Iran | 2011 | Consecutive | 20% | not |
| and Somi | | | | | patients recruited | | specified |
| 2013) | | | | | from university | | |
| | | | | | clinic | | |
| (Fujino, | AIH | 102 | Japan | 2016- | Patients with | 32.4% any | NRS |
| Tanaka | | | | 2017 | chronic liver disease | severity | |
| et al. 2019) | | | | | attending Hiroshima | ~18% | |
| | | | | | University Hospital | moderate- | |
| | | | | | | severe | |
| (Kido- | AIH | 11 | Japan | 2015- | patients who visited | 54.5% any | VAS and |
| Nakahara, | | | | 2016 | departments of | severity | VRS |
| Nakahara | | | | | internal medicine at | | |
| et al. 2019) | | | | | 3 general hospitals | | |
| (Oeda, | AIH | 70 | Japan | 2016 | outpatients from 9 | 24.3% any | VAS |
| Takahashi | | | | | facilities | severity | |
| et al. 2018) | | | | | | | |
| (Werner, | AIH | 473 | Sweden | <2003 | cohort of AIH | 4% (5 yrs | From |
| Wallerstedt | | | | | | post dx) | medical |
| et al. 2010) | | | | | | 14% (at | records |
| | | | | | | diagnosis) | |
| | | | | | | 22% (ever) | |
| (Koay, Lin | AIH type 1 | 48 | Taiwan | 2000- | type 1 AIH | 10.40% | not |
| et al. 2006) | | | | 2004 | diagnosed at a | | specified |
| | | | | | tertiary referral | | |
| | | | | | centre | | |
| (Gunsar, | AIH | 81 | Turkey | not | Hepatology clinic | 7.4% any | not |
| Gursel | | | | specified | | severity | specified |
| et al. 2016) | | | | | | | |
| (Gordon, | AIH | 1267 | UK | 2007- | adults dx with AIH | [42.4]% itch | not |
| Adhikary | | | | 2013 | across 28 hospitals | OR jaundice | specified |
| et al. 2018) | | | | | | | |
| (Perryman, | AIH | 142 | USA | 2014 | AIH patients 18 | 42% significant | not |
| Sreepati | | | | | years or older and | itch over past | specified |
| et al. 2014) | | | | | diagnosed by a | month | |
| | | | | | physician. | | |
| (Yerragorla, | AIH (non - | 5 | USA | 2001- | New York, Harlem - | 20% | not |
| Nekkalapudi | Caucasian) | | | 2017 | medical record | | specified |
| et al. 2017) | | | | | review | | |

NRS: numerical rating scale - 0-10 scale ranges from no itch (0) to worst imaginable itch (10). Itch of any severity defined as NRS ≥1, moderate-severe itch as NRS ≥4
VAS: Visual analogue scale - 10-cm-long horizontal line ranges from “no itch” (0 cm) to “worst imaginable itch” (10 cm)
VRS: Verbal rating scale consists of a list of phrases that describe increasing levels of pruritus intensity: no pruritus, mild pruritus, moderate pruritus, severe pruritus, and very severe pruritus.

### Biomarkers in Pruritus: Bile Acids and Autotaxin

### Bile Acids

Bile acids (BAs) are a major component of bile and play an essential role in emulsification and absorption of dietary fats and fat-soluble vitamins. Bile acids are also signalling molecules that are involved in regulation of their own biosynthesis as well as energy, glucose, and lipid metabolism by acting on various BA receptors. The liver synthesizes the primary BAs cholic acid and chenodeoxycholic acid which are readily conjugated with glycine or taurine. The secondary BAs deoxycholic acid and lithocholic acid, and tertiary bile acids such as ursodeoxycholic acid, are derived from primary BAs in the large intestine by the action of enzymes produced by commensal bacteria and may also be converted to glycine or taurine conjugates. The resulting BA pool, consisting of primary, secondary, conjugated and unconjugated BA species is optimally balanced to facilitate multiple biological functions. The BA pool composition can be represented by numerous methods including, but not limited to total serum bile acid (TSBA); total primary conjugated BAs, total secondary conjugated BAs, total primary BAs, total secondary BAs (Adams 2020 Liv Internat 40, 1356), molar concentration ratios of total primary to total secondary BAs (Jiao 2018 Gut, 67, 1881-1891); molar concentration ratios of total conjugated to total unconjugated BAs (Puri 2018 Hepatol 67(2) 534; Chen 2020 Clin Rev Allergy & Immunol, 58, 25-38); molar concentration ratios of total cholic acid to total chenodeoxycholic acid (Jurate 2017 16(4) 569-573); molar concentration ratios of total glycine conjugates to total taurine conjugates (Yara 2019 GastroHep, 1, 302-310); wherein ‘total’ is the sum of multiple unconjugated (parent) and/or conjugated BAs [Fiorucci, et al. Bile acids and their receptors in metabolic disorders. Progress in Lipid Research, 2021, 82, 101094].

The BA pool is efficiently utilized and recycled by a process known as enterohepatic recirculation, such that BAs travel from the liver through the bile and gallbladder into the small intestine. The primary and conjugated BAs are then mostly absorbed in the terminal ileum by the action of the ileal bile acid transporter (IBAT), also known as apical sodium-dependent bile acid transporter (ASBT), whereas secondary and unconjugated BAs are passively absorbed in the colon. Following intestinal absorption, the BAs are transported back to the liver via the hepatic portal vein and various other transporters. [Di Caula, et al., Bile Acid Physiology, Ann. Hepatology 2017, 16(S1), s4-s14.]

In cholestatic conditions the transit of BAs from liver to intestine via the bile and gallbladder is impeded, resulting in a build-up of BAs in the liver thus causing inflammation and injury. Under these conditions, the liver compensates by upregulating BA transporters responsible for efflux and downregulating BA transporters responsible for uptake and downregulating the biosynthesis of new BAs [Langedijk, J A, et al., Cholestasis-Associated Pruritus and its Pruritogens. Frontiers in Medicine, 2021, 8. 639674]. These concerted actions result in atypically high BA concentration in the systemic circulation, typically reported as total serum bile acids (TSBA). Since primary and conjugated BAs are the predominant BA components in the liver, and because these are preferentially impacted by the change in hepatocyte transporters during cholestasis, the systemic BA pool (i.e. serum concentration of all BAs) is preferentially enriched in primary and conjugated BAs in patients with cholestasis. Normally, in a healthy individual, serum concentration of BAs or TSBA, is low because BAs are effectively restricted to enterohepatic recirculation.

The increase in BAs in the body, as measured by serum concentration, is postulated to be one of several mediators of cholestatic pruritus, though the lack of correlation of serum bile acid levels with cholestatic itch suggests other factors may also contribute to pruritus. [Lindor 2019 Hepatology 69(1)].

Inhibition of IBAT by the inhibitor molecule linerixibat in adult PBC patients results in lowered conjugated and total serum BAs, by preferential and increased clearance of primary and conjugated BAs through the intestine into the faeces, thereby lowering the overall burden of high BA concentrations in the enterohepatic recirculation and associated spill-over into the systemic circulation of patients with cholestatic pruritus [Hegade et al. Lancet 2017, 389, 1114-1123. Hegade et al. Liver International, 2019, 39, 967-975]. In addition, differences in TSBA and BA pool composition have been associated with severity of PBC and NAFLD (or MASLD) [Chen et al Clin Revs Allergy & Immunology 2020 February; 58(1):25-38, Caussey et al. Gut 2019; 68(10): 1884-1892).

Clinical and biochemical symptoms of intrahepatic cholestasis of pregnancy (ICP), which is the most common hepatic disorder related to pregnancy in women, include pruritus and increased levels of TSBA and autotaxin [Piechota J. Clin. Med. 2020, 9, 1361. Journal of Hepatology 2015 vol. 62 j 897-904]. In well-recognized cholestatic disorders, both TSBA and BA pool composition are frequently measured as the levels of individual BAs or the ratio of primary to secondary BA or conjugated to unconjugated BAs can be preferentially impacted during cholestasis.

In the serum of patients with cholestatic liver disease who report severe, generalized pruritus, numerous possible pruritogens are found to be increased, including BAs [PNAS 2019, 116 (21); 10525-10530]. TBSAs are found to be elevated in NASH (or MASH) compared to healthy subjects and this is due to the increase in conjugated and primary BAs [Dig Dis Sci, 2015; 60: 3318-3328. Gastro Hep. 2019; 1:302-310]. NASH (or MASH) is associated with elevated and significantly altered circulating BA composition compared to healthy controls [Puri P, Daita K, Joyce A, et al. ‘The presence and severity of non-alcoholic steatohepatitis is associated with specific changes in circulating bile acids’. Hepatology. 2018; 67(2):534-548]. Although elevated and altered BA composition has been observed in NASH (or MASH), to date it has not been associated with pruritus. Example 4 herein now newly demonstrates that increases of TSBA in NAFLD (or MASLD), particularly NASH (or MASH) correlate with reported itch severity.

In chronic HepB infection TBSAs are not found to be increased, but specific individual BAs are increased leading to an increase in the ratio of primary to secondary BAs and conjugated to unconjugated BAs [Sun et al., Frontiers in Medicine October 2021; Volume 8: Article 708495].

Autoimmune Hepatitis (AIH) is an autoimmune liver disease (AILD). TSBA and specific conjugated bile acids were found to be elevated in a Chinese AIH cohort [Lian et al., Hepatobiliary Pancreat Dis Int 2015 (August 15), Vol 14 (4): 413-421].

### Autotaxin

Autotaxin (ATX) is a secreted enzyme that converts lysophosphatidylcholine to lysophosphatidic acid [Lindor 2019 Hepatology 69(1); J Biol Chem. 2002; 277: 39436-39442 Hepatology. 2012; 56: 1391-1400]. Serum ATX levels are found to be prominently increased in patients with cholestatic liver disease reporting pruritus, as compared to those patients without pruritus [Frontiers in Medicine 2021; Volume 8: Article 639674]. ATX activity correlates significantly with intensity of pruritus but it is predominantly increased in pruritus of cholestasis and not in pruritus of other origins [Gastroenterology 2010; 139:1008-18; Hepatology 2012; 56:1391-400]. Although ATX is not excreted into bile [Gastroenterology 2010; 139:1008-1848], interruption of the enterohepatic circulation, e.g. by nasobiliary drainage and IBAT inhibition, decreases both circulating ATX levels and pruritus scores [Gastroenterology 2010; 139:1008-1848. Sci Rep. 2018 8:6658. Lancet 2017; 389:1114-23. Liver Int. 2019; 39:967-75].

Inhibition of IBAT by linerixibat in adult PBC patients resulted in lowered serum levels of autotaxin. (Hegade 2017 Lancet, 389, 1114-1123; Levy 2022) In addition, a correlation between the % change from baseline in ATX and the % change from baseline in some serum bile acids was reported [Hegade et al. Liver International 2019, 39, 967-975] but no significant correlations were seen between serum BAs, autotaxin and baseline pruritus scores, and reductions in serum BAs did not correlate with reductions in pruritus scores.

### IBAT Inhibitors

In cholestatic pruritus associated with cholestatic liver diseases such as PBC and PSC, attention has turned to the ileal bile acid transporter (IBAT), also known as apical sodium-dependent bile acid transporter (ASBT), as a therapeutic target.

As mentioned hereinabove, the molecular IBAT inhibitors maralixibat (LIVMARLI) and odevixibat (BYLVAY) were recently approved by the FDA for the treatment of cholestatic pruritus in Alagille syndrome and pediatric familial intrahepatic cholestasis (PFIC), respectively (https://www.accessdata.fda.gov/drugsatfda_docs/label/2021/214662s000lbl.pdf
https://www.accessdata.fda.gov/drugsatfda_docs/label/2021/215498s000lbl.pdf).

Odevixibat was also approved by the European Medicines Agency:
(https://www.ema.europa.eu/en/documents/product-information/bylvay-epar-product-information_en.pdf).

Given that the present disclosure herein, particularly Examples 1-3, supports the fact that the pruritus experienced by patients with CLDs such as HepB, HepC, AIH and NAFLD (or MASLD), for example NASH (or MASH), resembles that of cholestatic itch and thus may be potentially cholestatic in nature or bile-acid induced, the pruritus is expected to be treatable with a therapy known to treat pruritus in cholestatic liver diseases, for example treatment of the pruritus with an IBAT inhibitor.

In addition, given that serum BAs are found to be raised in NAFLD (or MASLD) and NASH (or MASH), and Example 4 now newly demonstrates that increases of TSBA in NAFLD (or MASLD), particularly NASH (or MASH) correlate with reported itch severity, the pruritus is expected to be treatable with a therapy known to reduce bile acid levels, for example treatment of the pruritus with an IBAT inhibitor.

Therefore, in one aspect of the invention there is provided an IBAT inhibitor for use in the treatment of pruritus in NAFLD (or MASLD). In another aspect there is provided a method of treatment of pruritus in NAFLD (or MASLD) in a human in need thereof, comprising administering to said human a therapeutically effective amount of an IBAT inhibitor.

In another aspect of the invention there is provided an IBAT inhibitor for use in the treatment of cholestatic pruritus in NAFLD (or MASLD). In a further aspect there is provided a method of treatment of cholestatic pruritus in NAFLD (or MASLD) in a human in need thereof, comprising administering to said human a therapeutically effective amount of an IBAT inhibitor.

In a further aspect of the invention there is provided an IBAT inhibitor for use in the treatment of pruritus in HepB, for example cholestatic pruritus in HepB. In a further aspect there is provided a method of treatment of pruritus in HepB, for example cholestatic pruritus in HepB in a human in need thereof, comprising administering to said human a therapeutically effective amount of an IBAT inhibitor.

In a further aspect of the invention there is provided an IBAT inhibitor for use in the treatment of pruritus in HepC, for example cholestatic pruritus in HepC. In a further aspect there is provided a method of treatment of pruritus in HepC, for example cholestatic pruritus in HepC, in a human in need thereof, comprising administering to said human a therapeutically effective amount of an IBAT inhibitor.

In a further aspect of the invention there is provided an IBAT inhibitor for use in the treatment of pruritus in AIH for example cholestatic pruritus in AIH. In a further aspect there is provided a method of treatment of pruritus in AIH, for example cholestatic pruritus in AIH in a human in need thereof, comprising administering to said human a therapeutically effective amount of an IBAT inhibitor.

In one aspect of the invention there is provided the use of an IBAT inhibitor in the manufacture of a medicament for use in the treatment of pruritus in NAFLD (or MASLD). In another aspect there is provided the use of an IBAT inhibitor in the manufacture of a medicament for use in the treatment of cholestatic pruritus in NAFLD (or MASLD).

In one embodiment, the IBAT inhibitor is an antibody. The term “antibody” is used herein in the broadest sense to refer to molecules with an immunoglobulin-like domain (for example IgG, IgM, IgA, IgD or IgE) and includes monoclonal, recombinant, polyclonal, chimeric, human, humanized, multispecific antibodies, including bispecific antibodies, and heteroconjugate antibodies; a single variable domain (e.g., a domain antibody (DAB)), antigen binding antibody fragments, Fab, F(ab′)2, Fv, disulphide linked Fv, single chain Fv, disulphide-linked scFv, diabodies, TANDABS, etc. and modified versions of any of the foregoing (for a summary of alternative “antibody” formats see Holliger and Hudson, Nature Biotechnology, 2005, Vol 23, No. 9, 1126-1136).

In one embodiment, the IBAT inhibitor is a small molecule. In one embodiment, the IBAT inhibitor for use in the present invention is selected from: maralixibat, volixibat, odevixibat, elobixibat, linerixibat, Albireo A-3907 and CJ-14199 (by CJ Healthcare); or a pharmaceutically acceptable salt, solvate or crystalline form thereof. In another embodiment, the IBAT inhibitor for use in the present invention is selected from: maralixibat, volixibat, odevixibat, elobixibat, linerixibat and Albireo A-3907; or a pharmaceutically acceptable salt or crystalline form thereof.

In one embodiment, the IBAT inhibitor for use in the present invention, or the IBAT inhibitor used in the methods of treatment of the present invention, is present as the free acid. In another embodiment, IBAT inhibitor for use in the present invention is present in salt form. For example, maralixibat may be present in salt form, e.g., maralixibat chloride.

In a further embodiment, the IBAT inhibitor for use in the present invention, or the IBAT inhibitor used in the methods of treatment of the present invention, is present in crystalline form.

IBAT inhibitors, for example linerixibat, have been shown to reduce serum bile acid concentration. We now newly show in Example 5 that linerixibat treatment results in a decrease in total serum bile acids which correlates with itch reduction in patients with cholestatic pruritus in PBC [Karatza et al., EASL abstract: Karatza E, et al. J Hepatol 2023:78 (S1):S367; poster: Karatza et al., EASL poster 2023 https://www.postersessiononline.eu/173580348_eu/congresos/ILC2023/aula/-TOP_62_ILC2023.pdf; manuscript: https://doi.org/10.1111/liv.15982]. We also now show in Example 4 that bile acids increase with itch severity in NAFLD (or MASLD), including NASH (or MASH). Therefore, treatment of a patient experiencing or suffering from pruritus in NAFLD (or MASLD), including NASH (or MASH), with an IBAT inhibitor such as linerixibat is expected to reduce serum bile acid concentration and in turn reduce itch severity.

Accordingly, in one embodiment of the invention there is provided an IBAT inhibitor for use in the treatment of pruritus in NASH (or MASH), for example cholestatic pruritus in NASH (or MASH). In another embodiment there is provided a method of treatment of pruritus in NASH (or MASH), for example cholestatic pruritus in NASH (or MASH), in a human in need thereof, comprising administering to said human a therapeutically effective amount of an IBAT inhibitor.

In another embodiment of the invention there is provided an IBAT inhibitor for use in the treatment of pruritus in NAFL, for example cholestatic pruritus in NAFL. In a further embodiment there is provided a method of treatment of pruritus in NAFL, for example cholestatic pruritus in NAFL, in a human in need thereof, comprising administering to said human a therapeutically effective amount of an IBAT inhibitor.

In another embodiment of the invention there is provided an IBAT inhibitor for use in the treatment of pruritus in NAFLD (or MASLD), for example cholestatic pruritus in NAFLD (or MASLD). In a further embodiment there is provided a method of treatment of pruritus in NAFLD (or MASLD), for example cholestatic pruritus in NAFLD (or MASLD), in a human in need thereof, comprising administering to said human a therapeutically effective amount of an IBAT inhibitor.

In one embodiment of the invention there is provided the use of an IBAT inhibitor in the manufacture of a medicament for use in the treatment of pruritus in NASH (or MASH). In another aspect there is provided the use of an IBAT inhibitor in the manufacture of a medicament for use in the treatment of cholestatic pruritus in NASH (or MASH).

In another embodiment of the invention there is provided the use of an IBAT inhibitor in the manufacture of a medicament for use in the treatment of pruritus in NAFL. In another aspect there is provided the use of an IBAT inhibitor in the manufacture of a medicament for use in the treatment of cholestatic pruritus in NAFL.

In another embodiment of the invention there is provided the use of an IBAT inhibitor in the manufacture of a medicament for use in the treatment of pruritus in NAFLD (or MASLD). In another aspect there is provided the use of an IBAT inhibitor in the manufacture of a medicament for use in the treatment of cholestatic pruritus in NAFLD (or MASLD).

In a further embodiment of the invention there is provided an IBAT inhibitor for use in the treatment of pruritus in noncirrhotic NASH (or MASH), for example cholestatic pruritus in noncirrhotic NASH (or MASH). In a further embodiment there is provided a method of treatment of pruritus in noncirrhotic NASH (or MASH), for example cholestatic pruritus in noncirrhotic NASH (or MASH), in a human in need thereof, comprising administering to said human a therapeutically effective amount of an IBAT inhibitor.

In a further embodiment of the invention there is provided an IBAT inhibitor for use in the treatment of pruritus in NASH (or MASH) with cirrhosis, for example cholestatic pruritus in NASH (or MASH) with cirrhosis. In a further embodiment there is provided a method of treatment of pruritus in NASH (or MASH) with cirrhosis, for example cholestatic pruritus in NASH (or MASH), with cirrhosis in a human in need thereof, comprising administering to said human a therapeutically effective amount of an IBAT inhibitor.

### Linerixibat

An example of an IBAT inhibitor, linerixibat, is a minimally absorbed oral small molecule, showed significant improvements in pruritus versus placebo in a phase 2a study of patients with PBC and reduces absorption of bile acids in the terminal ileum and increases faecal bile acid excretion [Al-Dury S, Marschall H U. Front Pharmacol 2018; 9:931 and Hegade V S, et al. Lancet 2017; 389:1114-23]. The phase 2b GLIMMER study (NCT02966834) is the largest randomized investigational study of PBC patients with cholestatic pruritus to date. In this study, linerixibat dose-dependently ameliorated pruritus over 12 weeks of treatment, indicating the potential of linerixibat as a future treatment option for cholestatic pruritus in PBC (Clin Gastroenterol Hepatol. 2022; in press).

Linerixibat is the INN and USAN name for this compound, which is also known as GSK2330672, sometimes abbreviated as GSK672. The IUPAC name for the compound is rac-3-((((3R,5R)-3-butyl-3-ethyl-7-methoxy-1,1-dioxido-5-phenyl-2,3,4,5-tetrahydrobenzo[f][1,4]thiazepin-8-yl)methyl)amino)pentanedioic acid. It has the structure:

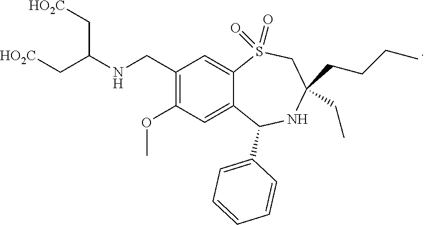 [SMILES: CCCC[C@]1(CC)CS(=O)(=O)c2cc(CNC(CC(=O)O)CC(=O)O)c(OC)cc2[C@@H](c2ccccc2)N1]

Patent publication WO 2011/137135 discloses linerixibat among other compounds. WO 2011/137135 also discloses methods of synthesis of the compound.

The preparation of linerixibat is also disclosed in J. Med. Chem, Vol 56, pp 10 5094-5114 (2013), in J. Org. Chem., Vol 78, pp 12726-12734 (2013) and in patent publications WO 2016/020785 and WO 2018/002827.

Therefore, in one embodiment there is provided an IBAT inhibitor for use in the present invention, or in a method of treatment of the present invention, wherein the IBAT inhibitor is linerixibat or a pharmaceutically acceptable salt or crystalline form thereof.

In another embodiment there is provided an IBAT inhibitor for use in the present invention, or in a method of treatment of the present invention, wherein the IBAT inhibitor is linerixibat free acid.

Linerixibat has been demonstrated to be generally well tolerated in clinical studies to date, within both the type 2 diabetes mellitus (T2DM) population [Nunez D J, et al. Diabetes Obes Metab 2016; 18(7):654-662] as well as in the PBC population which have included compensated cirrhotic patients [Levy 2022 Clin Gastroenterol and Hep, online: https://doi.org/10.1016/j.cgh.2022.10.032].

Potential metabolic effects of linerixibat that may be of value to NASH (or MASH) patients include reductions in fasting glucose and LDL cholesterol as demonstrated in the T2DM population.

In both of the Phase 2 clinical studies of PBC patients with cholestatic pruritus, namely the Phase 2a study (Hegade, Kendrick et al. 2017) and the Phase 2b study GLIMMER (NCT02966834, results online: Levy 2002 https://doi.org/10.1016/j.cgh.2022.10.032), Linerixibat reduced pruritus, as well as serum concentrations of both TSBA and autotaxin/autotaxin activity, consistent with the purported role of these biomarkers in pruritus. Upon withdrawal of linerixibat, there was evidence of return of itch, as well as a change of TSBA and autotaxin levels and autotaxin activity, towards baseline levels.

In Example 5 herein we now demonstrate that change in TSBA over the study treatment period correlates significantly with, and can be predictive of, improvement in itch in patients with PBC. In Example 6 herein we now demonstrate that serum ATX and FGF-19 may function as biomarkers of itch response to linerixibat treatment, and that reductions in these biomarkers are associated with clinical response in patients with PBC and pruritus.

Accordingly, in a further embodiment of the invention there is provided linerixibat or a pharmaceutically acceptable salt or crystalline form thereof for use in the treatment of pruritus in NASH (or MASH), for example cholestatic pruritus in NASH (or MASH). In another embodiment there is provided a method of treatment of pruritus in NASH (or MASH), for example cholestatic pruritus in NASH (or MASH), in a human in need thereof, comprising administering to said human a therapeutically effective amount of or a pharmaceutically acceptable salt or crystalline form thereof. In one embodiment of the invention there is provided the use of linerixibat in the manufacture of a medicament for use in the treatment of pruritus, for example cholestatic pruritus, in NASH (or MASH).

In a yet further embodiment of the invention there is provided linerixibat or a pharmaceutically acceptable salt or crystalline form thereof for use in the treatment of pruritus in NAFL, for example cholestatic pruritus in NAFL. In another embodiment there is provided a method of treatment of pruritus in NAFL, for example cholestatic pruritus in NAFL, in a human in need thereof, comprising administering to said human a therapeutically effective amount of or a pharmaceutically acceptable salt or crystalline form thereof. In one embodiment of the invention there is provided the use of linerixibat in the manufacture of a medicament for use in the treatment of pruritus, for example cholestatic pruritus, in NAFL.

In a yet further embodiment of the invention there is provided linerixibat or a pharmaceutically acceptable salt or crystalline form thereof for use in the treatment of pruritus in NAFLD (or MASLD), for example cholestatic pruritus in NAFLD (or MASLD). In another embodiment there is provided a method of treatment of pruritus in NAFLD (or MASLD), for example cholestatic pruritus in NAFLD (or MASLD), in a human in need thereof, comprising administering to said human a therapeutically effective amount of or a pharmaceutically acceptable salt or crystalline form thereof. In one embodiment of the invention there is provided the use of linerixibat in the manufacture of a medicament for use in the treatment of pruritus, for example cholestatic pruritus, in NAFLD (or MASLD).

In a further embodiment of the invention there is provided linerixibat or a pharmaceutically acceptable salt or crystalline form thereof for use in the treatment of pruritus in noncirrhotic NASH (or MASH), for example cholestatic pruritus in noncirrhotic NASH (or MASH). In another embodiment there is provided a method of treatment of pruritus in noncirrhotic NASH (or MASH), for example cholestatic pruritus in noncirrhotic NASH (or MASH), in a human in need thereof, comprising administering to said human a therapeutically effective amount of or a pharmaceutically acceptable salt or crystalline form thereof. In one embodiment of the invention there is provided the use of linerixibat in the manufacture of a medicament for use in the treatment of pruritus, for example cholestatic pruritus, in noncirrhotic NASH (or MASH).

In a further embodiment of the invention there is provided linerixibat or a pharmaceutically acceptable salt or crystalline form thereof for use in the treatment of pruritus in NASH (or MASH) with cirrhosis, for example cholestatic pruritus in NASH (or MASH) with cirrhosis. In another embodiment there is provided a method of treatment of pruritus in NASH (or MASH) with cirrhosis, for example cholestatic pruritus in NASH (or MASH) with cirrhosis, in a human in need thereof, comprising administering to said human a therapeutically effective amount of or a pharmaceutically acceptable salt or crystalline form thereof. In one embodiment of the invention there is provided the use of linerixibat in the manufacture of a medicament for use in the treatment of pruritus, for example cholestatic pruritus, in NASH (or MASH) with cirrhosis.

Linerixibat can exist in amorphous or crystalline forms, disclosed in patent publication WO 2011/137135 and patent application PCT/EP2021/078734. Accordingly, in one embodiment the IBAT inhibitor for use in the present invention, or in a method of treatment of the present invention, is linerixibat in crystalline form.

In one embodiment, the crystalline form of linerixibat may comprise one or more polymorphic crystalline forms. Linerixibat may be present in crystalline form I as disclosed in WO 2011/137135 and patent application PCT/EP2021/078734.

In one embodiment, linerixibat is present in a mixture of crystalline forms I and III as disclosed in patent application PCT/EP2021/078734. In another embodiment, linerixibat is in amorphous form.

### PPAR Agonists

PPAR (peroxisome proliferator-activated receptor) agonists include selective activators of PPAR alpha, PPAR delta, and/or activators of both alpha and delta PPAR isoforms, and/or non-selective so-called “pan”-PPAR activators.

PPAR agonists have the potential to lower serum bile acids and reduce itch in PBC. PPAR agonists include, but are not limited to, seladelpar, elafibranor, bezafibrate, and lanifibranor.

Therefore, treatment of a patient experiencing or suffering from pruritus in NAFLD (or MASLD), including NASH (or MASH), with a PPAR agonist which reduces serum bile acid concentration, may reduce itch severity.

Accordingly, in one embodiment of the invention there is provided a PPAR agonist for use in the treatment of pruritus in NAFLD (or MASLD), for example treatment of cholestatic pruritus in NAFLD (or MASLD), or treatment of pruritus or cholestatic pruritis in NASH (or MASH).

In another embodiment of the invention there is provided a method of treatment of pruritus in NAFLD (or MASLD) in a human in need thereof, comprising administering to said human a therapeutically effective amount of an PPAR agonist. In certain embodiments of such method, said treatment of pruritus in NAFLD (or MASLD) is the treatment of cholestatic pruritus in NAFLD (or MASLD) or the treatment of pruritus or cholestatic pruritis in NASH (or MASH).

In a further aspect of the invention there is provided an PPAR agonist for use in the treatment of pruritus in HepB, for example cholestatic pruritus in HepB. In a further aspect there is provided a method of treatment of pruritus in HepB, for example cholestatic pruritus in HepB in a human in need thereof, comprising administering to said human a therapeutically effective amount of an PPAR agonist.

In a further aspect of the invention there is provided an PPAR agonist for use in the treatment of pruritus in HepC, for example cholestatic pruritus in HepC. In a further aspect there is provided a method of treatment of pruritus in HepC, for example cholestatic pruritus in HepC, in a human in need thereof, comprising administering to said human a therapeutically effective amount of an PPAR agonist.

In a further aspect of the invention there is provided an PPAR agonist for use in the treatment of pruritus in AIH, for example cholestatic pruritus in AIH. In a further aspect there is provided a method of treatment of pruritus in AIH, for example cholestatic pruritus in AIH, in a human in need thereof, comprising administering to said human a therapeutically effective amount of an PPAR agonist.

### FGF-19 Analogues

FGF-19 (serum fibroblast growth factor-19) analogues, such as aldafermin, are known to suppress C4 and/or serum bile acid levels in NASH (or MASH) and PSC patients [Harrison 2019 Hepatology; Sanyal 2021 JHEP Reports].

Therefore, treatment of a patient experiencing or suffering from pruritus in NAFLD (or MASLD), including NASH (or MASH), with an FGF-19 analogue is expected to reduce serum bile acid concentration and in turn may reduce itch severity.

Accordingly, in one embodiment of the invention there is provided an FGF-19 analogue for use in the treatment of pruritus in NAFLD (or MASLD), for example treatment of cholestatic pruritus in NAFLD (or MASLD) or treatment of pruritus in NASH (or MASH).

In another embodiment of the invention there is provided a method of treatment of pruritus in NAFLD (or MASLD) in a human in need thereof, comprising administering to said human a therapeutically effective amount of an FGF-19 analogue. In certain embodiments of such method, said treatment of pruritus in NAFLD (or MASLD) is the treatment of cholestatic pruritus in NAFLD (or MASLD) or the treatment of pruritus in NASH (or MASH).

In a further aspect of the invention there is provided an FGF-19 analogue for use in the treatment of pruritus in HepB, for example cholestatic pruritus in HepB. In a further aspect there is provided a method of treatment of pruritus in HepB, for example cholestatic pruritus in HepB in a human in need thereof, comprising administering to said human a therapeutically effective amount of an FGF-19 analogue.

In a further aspect of the invention there is provided an FGF-19 analogue for use in the treatment of pruritus in HepC, for example cholestatic pruritus in HepC. In a further aspect there is provided a method of treatment of pruritus in HepC, for example cholestatic pruritus in HepC, in a human in need thereof, comprising administering to said human a therapeutically effective amount of an FGF-19 analogue.

In a further aspect of the invention there is provided an FGF-19 analogue for use in the treatment of pruritus in AIH, for example cholestatic pruritus in AIH. In a further aspect there is provided a method of treatment of pruritus in AIH, for example cholestatic pruritus in AIH in a human in need thereof, comprising administering to said human a therapeutically effective amount of an FGF-19 analogue.

### Discussion of Examples

As mentioned hereinabove, treatment of the underlying CLD condition does not necessarily lead to a reduction of pruritus and in some cases treatment of the underlying disease results in treatment emergent pruritus or an exacerbation of the associated pruritus.

In the natural history study described in Example 1 herein, data was collected in respect of itch experienced by patients with a broad range of chronic liver diseases. The results of this study support literature estimates in demonstrating that pruritus in NASH (or MASH) is prevalent, similar in nature to pruritus observed in cholestatic liver diseases, and is an unmet medical need. The results of this study also provide some supportive evidence for the potential cholestatic nature of pruritus in CLDs such as NAFLD (or MASLD), for example NASH (or MASH), a chronic liver disease which is not typically considered to be cholestatic. Therefore, pruritus in NASH (or MASH) may be amenable to treatment with an IBAT inhibitor.

Example 2a described herein is a comparison of the itch experienced in PBC, PSC, AIH, HepB, HepC, and NASH (or MASH) conditions in the natural history study of Example 1 with itch reported in the literature in conditions where the itch is known not to be cholestatic in nature (namely itch in a hemodialysis patient population, a patient population with atopic dermatitis and a clinical trial patient population with uremic pruritus). The comparison is shown in the form of a 5D-Itch Plot. It shows that pruritus in NASH (or MASH), AIH, HepB and HepC resembles pruritus in the known cholestatic liver diseases or in those with cholestatic features, with regard to degree and duration, on the 5D itch instrument. This provides further supportive evidence relating to the similarities between itch in known cholestatic liver diseases and itch in AIH, HepB, HepC and NASH (or MASH). Therefore, pruritus in AIH, HepB, HepC and/or NASH (or MASH) may be amenable to treatment with an IBAT inhibitor.

The PRO studies described in Example 3 and Example 3a provide evidence that the itch experienced by patients suffering from HepB, HepC, AIH, DILI, NASH (or MASH) or NAFL (or MASLD) is not qualitatively different from that in PSC or PBC, which are known cholestatic conditions. Therefore, pruritus in these conditions, particularly HepB, HepC, AIH, NASH (or MASH) and NAFL (or MASLD), may be amenable to treatment with an IBAT inhibitor.

Example 4 described herein is a study characterising the burden and clinical trajectory of pruritus in primary sclerosing cholangitis (PSC) and non-PBC/PSC chronic liver disease including NAFLD (or MASLD)/NASH (or MASH). The results of the study of Example 4 support the finding that bile acids and autotaxin increase with itch severity in NAFLD (or MASLD), including NASH (or MASH). As such, bile acids alone or together with autotaxin may play a role in the pathogenesis of pruritus in NASH (or MASH). Therefore, pruritus in NASH (or MASH) may be amenable to treatment with an IBAT inhibitor.

Example 5 herein is an analysis of the relationship between linerixibat dose and change in TSBA over time, and change in pruritus in subjects with PBC. We found that linerixibat treatment leads to rapid and dose-dependent reductions in TSBA. Baseline TSBA levels do not correlate with on-treatment change in NRS itch score, suggesting they do not predict linerixibat response. However, change in TSBA over the treatment period correlates significantly with, and can be predictive of, improvement in itch in patients with PBC.

Example 6 herein is an analysis of the association between itch response and changes in ATX and FGF-19 biomarker levels following linerixibat treatment in patients with cholestatic pruritus associated with PBC. We found that serum ATX and FGF-19 may function as biomarkers of itch response to linerixibat treatment. Reductions in these biomarkers are found to be associated with clinical response in patients with PBC and pruritus.

### Medical Use

In respect of an IBAT inhibitor, for example linerixibat or a pharmaceutically acceptable salt thereof, for use in treatment or in the methods of treatment as described herein:

In one embodiment, the IBAT inhibitor, for example linerixibator a pharmaceutically acceptable salt thereof, is administered in an amount of between 3 mg and 100 mg, twice daily, for example between 30 mg and 100 mg twice daily.

In one embodiment, the IBAT inhibitor, for example linerixibat or a pharmaceutically acceptable salt thereof, is administered in an amount of between 40 mg and 90 mg, twice daily.

In one embodiment, the IBAT inhibitor, for example linerixibat or a pharmaceutically acceptable salt thereof, is administered in an amount of approximately 90 mg, twice daily.

In one embodiment, the IBAT inhibitor, for example linerixibat or a pharmaceutically acceptable salt thereof, is administered in an amount of approximately 40 mg, twice daily.

### Pharmaceutical Compositions

In respect of an IBAT inhibitor, such as linerixibat or a pharmaceutically acceptable salt thereof, for use in treatment or in the methods of treatment as described herein:

In one embodiment, in respect of an IBAT inhibitor for use in treatment or in the methods of treatment as described herein, the IBAT inhibitor is administered orally.

In one embodiment, the IBAT inhibitor is formulated into a solid dosage form such as a capsule or tablet. In one embodiment, the IBAT inhibitor is formulated into a tablet. In one embodiment, the IBAT inhibitor is administered as an immediate release formulation such as an immediate release tablet. In another embodiment, the tablet further comprises filler, disintegrant, and lubricant. One example of a suitable tablet is a tablet comprising an IBAT inhibitor, microcrystalline cellulose, and magnesium stearate. Another example of a suitable tablet is a tablet comprising an IBAT inhibitor, microcrystalline cellulose, magnesium stearate and croscarmellose sodium.

In one embodiment the tablet comprises from 5 to 100 mg of an IBAT inhibitor. In one embodiment, the tablet comprises from 20 to 90 mg of an IBAT inhibitor. In another embodiment, the tablet comprises 80 mg of an IBAT inhibitor. In another embodiment, the tablet comprises 90 mg of an IBAT inhibitor. In another embodiment, the tablet comprises 45 mg of an IBAT inhibitor. In another embodiment, the tablet comprises 40 mg of an IBAT inhibitor. In another embodiment, the tablet comprises 20 mg of an IBAT inhibitor.

For the avoidance of doubt, it is noted that any particular dose can be administered in a single tablet or oral dosage form or multiple tablets or other oral dosage form. For example, a dose of 40 mg could be administered as a single 40 mg tablet, or two 20 mg tablets, or four 10 mg tablets, or eight 5 mg tablets, or for example a 20 mg and two 10 mg tablets.

It will be apparent that dose adjustments will result in the dose of the IBAT inhibitor being increased or decreased by one dose step at a time. Those receiving the highest (maximum) dose of the IBAT inhibitor who require a dose increase will maintain the same dose, while those receiving the lowest dose of the IBAT inhibitor that require a dose decrease will discontinue therapy with the IBAT inhibitor.

In a first aspect of the invention there is provided an IBAT inhibitor for use in the treatment of pruritus in NAFLD or MASLD. In another aspect there is provided an IBAT inhibitor for use in the treatment of pruritus in NAFLD. In a further aspect there is provided an IBAT inhibitor for use in the treatment of pruritus in MASLD.

In a second aspect of the invention there is provided an IBAT inhibitor for use in the treatment of cholestatic pruritus in NAFLD or MASLD. In another aspect there is provided an IBAT inhibitor for use in the treatment of cholestatic pruritus in NAFLD. In a further aspect there is provided an IBAT inhibitor for use in the treatment of cholestatic pruritus in MASLD.

In a third aspect of the invention there is provided a method of treatment of pruritus in NAFLD or MASLD in a human in need thereof, comprising administering to said human a therapeutically effective amount of an IBAT inhibitor. In another aspect there is provided a method of treatment of pruritus in NAFLD in a human in need thereof, comprising administering to said human a therapeutically effective amount of an IBAT inhibitor. In a further aspect there is provided a method of treatment of pruritus in MASLD in a human in need thereof, comprising administering to said human a therapeutically effective amount of an IBAT inhibitor.

In fourth aspect of the invention there is provided a method of treatment of cholestatic pruritus in NAFLD or MASLD in a human in need thereof, comprising administering to said human a therapeutically effective amount of an IBAT inhibitor. In another aspect there is provided a method of treatment of cholestatic pruritus in NAFLD in a human in need thereof, comprising administering to said human a therapeutically effective amount of an IBAT inhibitor. In a further aspect there is provided a method of treatment of cholestatic pruritus in MASLD in a human in need thereof, comprising administering to said human a therapeutically effective amount of an IBAT inhibitor.

In respect of one or more of the first, second, third and/or fourth aspects are included herein the following embodiments:

In one embodiment of the invention, the IBAT inhibitor is selected from: maralixibat, volixibat, odevixibat, elobixibat, linerixibat and Albireo A-3907; or a pharmaceutically acceptable salt or crystalline form thereof.

In another embodiment of the invention, the IBAT inhibitor is linerixibat or a pharmaceutically acceptable salt or crystalline form thereof.

In another embodiment of the invention, the IBAT inhibitor for use according to the first or second aspect, or the first embodiment, is linerixibat or a pharmaceutically acceptable salt or crystalline form thereof.

In another embodiment of the invention, the NAFLD or MASLD is NASH or MASH. In another embodiment, the NAFLD is NASH. In another embodiment, the MASLD is MASH. In a further embodiment, the NAFLD is NAFL.

In another embodiment of the invention, the NAFLD or MASLD is noncirrhotic NASH or MASH. In a further embodiment, the NAFLD is noncirrhotic NASH. In another embodiment, the MASLD is noncirrhotic MASH.

In another embodiment of the invention, the NAFLD or MASLD NASH or MASH with cirrhosis. In a further embodiment, the NAFLD is NASH with cirrhosis. In another embodiment, the MASLD is MASH with cirrhosis.

In another embodiment of the invention, the IBAT inhibitor is present as the free acid.

In another embodiment of the invention, the IBAT inhibitor is present in crystalline form.

In another embodiment of the invention, the pruritus is moderate to severe, for example ≥4 as measured on the NRS scale.

In another embodiment of the invention, the pruritus is moderate to severe, for example in a patient with cirrhosis having a TSBA level of about 34.

In another embodiment of the invention, the pruritus is moderate to severe, for example in a patient without cirrhosis having a TSBA level of about 11.

ABBREVIATIONS

| | |
|---|---|
| AIH | Autoimmune Hepatitis |
| ALD | Alcoholic Liver Disease |
| CLD | Chronic Liver Disease |
| DILI | Drug-Induced Liver Injury |
| HepB | Viral Hepatitis B |
| HepC | Viral Hepatitis C |
| MASLD | Metabolic dysfunction associated |
| | steatotic liver disease |
| MASH | Metabolic dysfunction-associated |
| | steatohepatitis |
| NAFL | non-alcoholic fatty liver (simple steatosis) |
| NAFLD | Non-Alcoholic Fatty Liver Disease |
| NASH | Non-Alcoholic Steatohepatitis |
| PBC | Primary Biliary Cholangitis (formerly |
| | Primary Biliary Cirrhosis) |
| PRO | Patient-reported Outcomes |
| PSC | Primary Sclerosing Cholangitis |
| TBSA | Total Serum Bile Acid |

### EXAMPLES

### Example 1

Natural History Study on the Health-Related Quality of Life of Liver Disease Patients with Pruritus

The overarching goal of this completed study was to characterize the natural history of pruritus and associated Health-Related Quality of Life (HRQoL) in patients with any of nine CLDs, namely those which are known to be cholestatic or can have cholestatic features (PBC, PSC, AIH, DILI) and other chronic liver diseases that are not typically considered to be cholestatic such as NAFLD (or MASLD), including NASH (or MASH) and NAFL, HepB, and HepC. To the best of our knowledge, this was the first global longitudinal study to compare pruritus in patients with these different liver diseases. This study had the following specific objectives:

### Primary Objectives

1. To prospectively describe patient-reported changes in pruritus, in terms of frequency, severity, location, timing, and its impact over a 6-month period;
2. To retrospectively describe the patient journey of pruritus, from its onset to present including diagnosis, treatment, severity, and impact.

### Secondary/Exploratory Objectives

1. To assess and describe differences in the retrospective and prospective course of pruritus between CLDs to determine whether the disease course, including progression in HRQoL, is similar across CLDs for planning of future clinical trials;
2. Assess prevalence of pruritus in CLDs based on screening questions.

The characteristics and nature of itch were gathered and evaluated in respect of certain of the nine CLDs, namely across some of the cholestatic CLDs and CLDs having cholestatic features (PBC, PSC, AIH) as compared with itch in patients with NASH (or MASH), HepB, HepC and DILI which are CLDs that are not typically considered to be cholestatic.

Patient experience, frequency, impact, and persistence of pruritus were examined by Worst Itch-NRS (WI-NRS) category (no itch 0, mild 1-3, moderate 4-6; severe 7-10).

### Example 1 Results

Support for the potential cholestatic nature of pruritus in NASH (or MASH), or that pruritus in NASH (or MASH) may be bile acid-induced, can be seen in data collected from this natural history study, which highlights the widespread experience of pruritus across NASH (or MASH) and various CLDs, as well as the prominence and debilitating burden of itch.

Focusing on NASH (or MASH), Table 1 illustrates that at screening, approximately 69% of NASH (or MASH) patients reported having pruritus with 46% reporting moderate to severe worst itch within the previous 3 months. Table 2 illustrates the frequency of experiencing itch ≥2 days in the last 2 weeks or scratching enough to make skin raw on on ≥2 days in the last 2 weeks by CLD. NASH (or MASH) patients with pruritus had a similar frequency of experiencing itch or scratching enough to make skin raw compared to patients with pruritus from cholestatic liver disease or a liver disease with cholestatic features (PBC, PSC and AIH), as well as patients with other CLDs and pruritus.

Table 3 below illustrates the similar qualitative nature of the type of itch experienced by patients with NASH (or MASH), and those with a cholestatic liver disease or with a liver disease having cholestatic features (PBC, PSC and AIH) when participants were asked to select which descriptors of itch applied to their experience of itch as a result of a chronic liver disease. Overall, the itch related to NASH (or MASH) appears to be similar to the other known cholestatic itch conditions with the majority of participants identifying the itch as a ‘deep’ and ‘urgent’ itch. Descriptors such as ‘bugs crawling’ and ‘relentless’ were also common experiences between diseases with itch that is known to be cholestatic and itch in NASH (or MASH). The type of itch was identified across all conditions as being different to the type of itch experienced with ‘hives’. Similarly, scratching did not alleviate the itch for a substantial proportion of patients across all liver diseases. Importantly, these descriptors of itch are similar to those used by patients with PBC in external, published studies (Vander Does, Levy et al. 2022; Rishe, Azarm et al. 2008).

Table 4 demonstrates that similar proportions of patients reported that their itch was worse mostly at night or equally during the day or night across liver diseases.

Similar to the factors reported to worsen itch in cholestatic liver disease described by Vander Does et al (Vander Does, Levy et al. 2022), Table 5 illustrates that heat, certain clothing and stress worsen itch in both patients with cholestatic liver disease (PBC, PSC, AIH) and in patients with NASH (or MASH) in the natural history study described above.

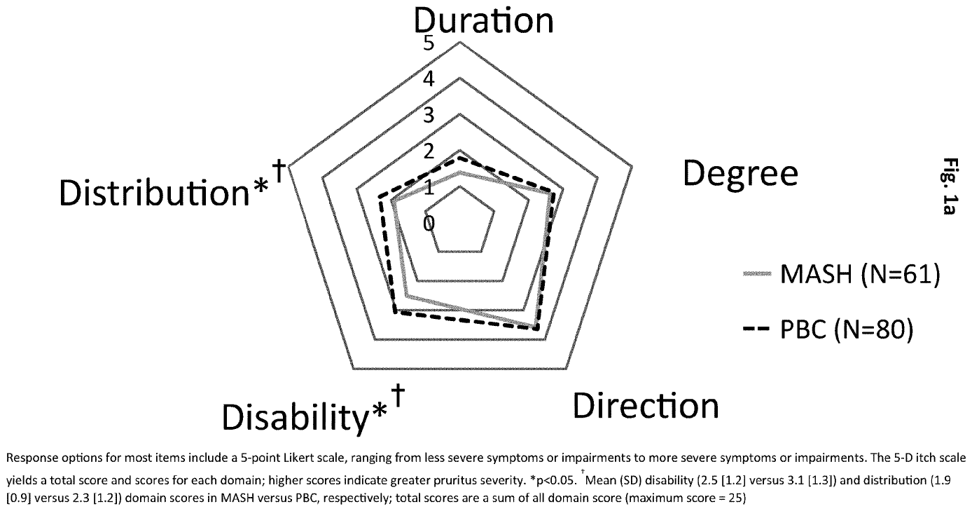

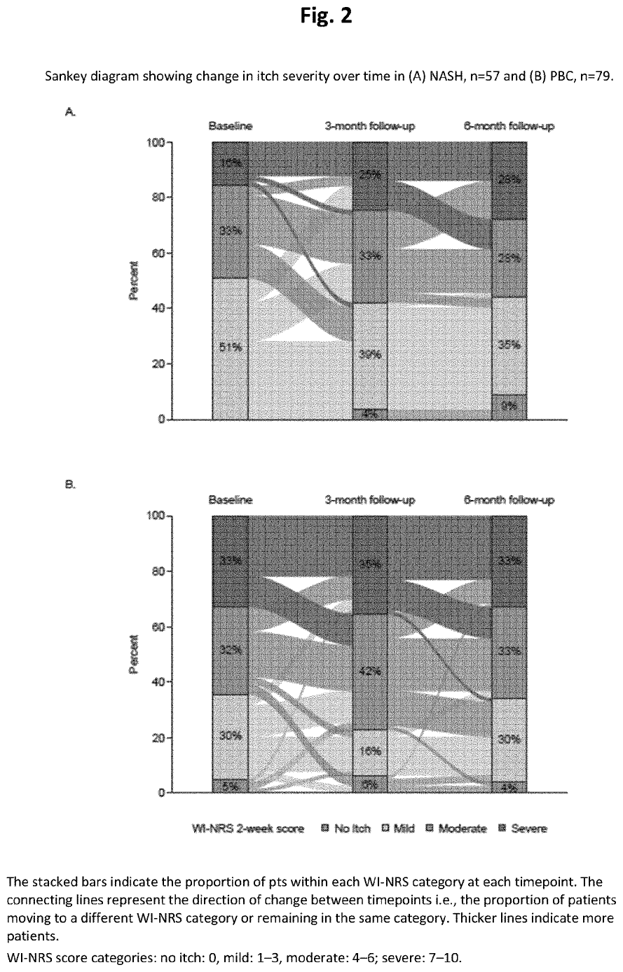

FIG. 2 illustrates that pruritus was largely persistent through 6 months of follow-up for NASH (or MASH) patients and PBC patients, particularly for those with moderate to severe itch.

Data collected from this study demonstrated that antihistamine and topical therapies are commonly used by patients with NASH (or MASH) to treat their pruritus, and although some improvement in pruritus is observed with these therapies, the reduction in pruritus was generally found to be insufficient, suggesting this patient population is actively seeking treatment for their itch.

### Example 1 Conclusions

This study found the patient experience of pruritus and its impact on QoL were comparable for NASH (or MASH) and PBC. Nearly half (46%) of screened patients with NASH (or MASH), and 39% of screened patients with PBC, reported moderate-to severe pruritus. Patients with NASH (or MASH) and PBC used similar language to describe pruritus. Pruritus persisted over the 6 month study timeframe for patients with both NASH (or MASH) and PBC. These data suggest that the evaluated attributes of itch in patients with NASH (or MASH) is not qualitatively different from attributes of itch in cholestatic liver diseases such as PBC and PSC and in liver diseases having cholestatic features. Therefore, pruritus in NASH (or MASH) may be amenable to treatment with an IBAT inhibitor.

TABLE 1
Screened patients reporting any pruritus or moderate to severe pruritus in the
previous 3 months; overall and across cholestatic (PBC, PSC, AIH) liver
diseases and patients with NASH (or MASH), Hep B, Hep C, or DILI

| | | Percentage of | | Percentage of | |
|---|---|---|---|---|---|
| | | patients with | | patients with | |
| | | pruritus, | | moderate to severe | |
| | | 3 month | | pruritus, 3 month | |
| | N | recall period, | | recall period, | |
| Cohort | screened | n (%)¹ | | n (%)² | |
| Overall | 717 | 403 | (56) | 302 | (42) |
| Autoimmune hepatitis (AIH) | 99 | 40 | (40) | 30 | (33) |
| Chronic viral hepatitis B (Hep B) | 50 | 35 | (70) | 26 | (52) |
| Chronic viral hepatitis C (Hep C) | 77 | 56 | (73) | 40 | (52) |
| Drug-induced liver injury (DILI) | 36 | 19 | (53) | 13 | (36) |
| Non-alcoholic steatohepatitis | 97 | 67 | (69) | 45 | (46) |
| (metabolic dysfunction- | | | | | |
| associated steatohepatitis) | | | | | |
| (NASH (or MASH)) | | | | | |
| Primary biliary cholangitis (PBC) | 174 | 85 | (49) | 68 | (39) |
| Primary sclerosing cholangitis (PSC) | 173 | 88 | (51) | 65 | (37) |

¹Derived based on screened patients reporting a WI-NRS score ≥1 (on a 0-10 scale) with a 3-month recall period.
²Derived based on screened patients reporting a WI-NRS score ≥4 (on a 0-10 scale) with a 3-month recall period.

TABLE 2
Enrolled patients experiencing itch ≥2 days in the last 2 weeks or scratching enough
to make skin raw on on ≥2 days in the last 2 weeks; overall and across cholestatic (PBC,
PSC, AIH) liver diseases and patients with NASH (or MASH), Hep B, Hep C, or DILI.

| | | | Experience |
|---|---|---|---|
| | | | of scratching |
| | | Experience | enough to |
| | | of itch | make skin raw |
| | | on ≥2 days | on ≥2 days |
| | | in the last | in the last |
| | N | 2 weeks, | two weeks, |
| Cohort | enrolled | n (%)¹ | n (%)¹ |
| Overall | 357 | 284 (80) | 96 (27) |
| Autoimmune hepatitis (AIH) | 36 | 30 (83) | 8 (22) |
| Chronic viral hepatitis B (Hep B) | 28 | 21 (75) | 7 (25) |
| Chronic viral hepatitis C (Hep C) | 54 | 49 (91) | 15 (28) |
| Drug-induced liver injury (DILI) | 19 | 18 (95) | 6 (32) |
| Non-alcoholic steatohepatitis (metabolic | 61 | 49 (80) | 10 (16) |
| dysfunction-associated steatohepatitis) | | | |
| (NASH (or MASH)) | | | |
| Primary biliary cholangitis (PBC) | 80 | 62 (78) | 27 (34) |
| Primary sclerosing cholangitis (PSC) | 74 | 52 (70) | 22 (30) |

¹Derived from 5D-D itch scale, n (%) based on enrolled patients.

TABLE 3
Patient experience of itch across cholestatic (PBC, PSC, AIH) liver
diseases and patients with NASH (or MASH), Hep B, Hep C, or DILI.

| Percentage of patients | | | | | | |
|---|---|---|---|---|---|---|
| NASH | | | | | | |
| (or MASH) | PBC | PSC | AIH | Hep B | Hep C | DILI |
| N = 61 | N = 80 | N = 74 | N = 36 | N = 28 | N = 54 | N = 19 |

In the time since your itch/pruritus began, how would
you describe the sensation of your itch/pruritus?

| | | | | | | | |
|---|---|---|---|---|---|---|---|
| Bugs crawling | 29.5 | 42.5 | 31.1 | 41.7 | 50.0 | 24.1 | 42.1 |
| Relentless | 44.3 | 45 | 51.4 | 30.6 | 21.4 | 38.9 | 42.1 |
| Want to tear my | 31.1 | 41.3 | 41.9 | 30.6 | 10.7 | 24.1 | 15.8 |
| skin off | | | | | | | |
| Prickly/ needles | 32.8 | 41.3 | 35.1 | 41.7 | 21.4 | 27.8 | 42.1 |
| Deep itch | 63.9 | 67.5 | 77 | 55.6 | 32.1 | 53.7 | 36.8 |
| Burning | 36.1 | 41.3 | 24.3 | 41.7 | 35.7 | 42.6 | 26.3 |
| Itch until I bleed | 21.3 | 45 | 51.4 | 22.2 | 14.3 | 18.5 | 26.3 |
| Hives | 8.2 | 13.8 | 12.2 | 5.6 | 3.6 | 7.4 | 5.3 |
| Urgent itch | 59 | 57.5 | 60.8 | 55.6 | 53.6 | 63.0 | 63.2 |
| Itch feels like it | 4.9 | 16.3 | 21.6 | 11.1 | 3.6 | 9.3 | 5.3 |
| is ‘in the veins' | | | | | | | |
| Scratching does | 45.9 | 51.3 | 44.6 | 30.6 | 14.3 | 29.6 | 10.5 |
| not help | | | | | | | |
| Other | 1.6 | 5 | 8.1 | 2.8 | 0.0 | 0.0 | 0.0 |

TABLE 4
Diurnal manifestations of itch across cholestatic (PBC, PSC, AIH) liver
diseases and patients with NASH (or MASH), Hep B, Hep C, or DILI.

| Percentage of patients | | | | | | |
|---|---|---|---|---|---|---|
| NASH | | | | | | |
| (or MASH) | PBC | PSC | AIH | Hep B | Hep C | DILI |
| N = 61 | N = 80 | N = 74 | N = 36 | N = 28 | N = 54 | N = 19 |

IN THE TIME SINCE YOUR ITCH/PRURITUS BEGAN, IN GENERAL, AT
WHAT TIME OF DAY HAS YOUR ITCH/PRURITUS BEEN THE WORST?

| | | | | | | | |
|---|---|---|---|---|---|---|---|
| DURING THE DAY | 32.8 | 23.8 | 21.6 | 30.6 | 50.0 | 53.7 | 57.9 |
| DURING THE NIGHT | 67.2 | 76.3 | 78.4 | 69.4 | 50.0 | 46.3 | 42.1 |
| OR ABOUT THE SAME | | | | | | | |
| DURING THE DAY/NIGHT | | | | | | | |

TABLE 5
Factors reported to worsen itch across cholestatic (PBC, PSC, AIH) liver
diseases and patients with NASH (or MASH), Hep B, Hep C, or DILI.

| Percentage of patients | | | | | | |
|---|---|---|---|---|---|---|
| NASH | | | | | | |
| (or MASH) | PBC | PSC | AIH | Hep B | Hep C | DILI |
| N = 61 | N = 80 | N = 74 | N = 36 | N = 28 | N = 54 | N = 19 |

In the time since your itch/pruritus began, please indicate how
much each of the following makes your itch/pruritus worse.

| | | | | | | | |
|---|---|---|---|---|---|---|---|
| Heat / warm weather | | | | | | | |
| Not at all | 34.4 | 32.5 | 17.6 | 47.2 | 42.9 | 31.5 | 26.3 |
| Slightly/somewhat worse | 34.4 | 50.1 | 54.1 | 30.6 | 32.2 | 35.2 | 47.4 |
| Much/very much worse | 31.2 | 17.5 | 28.4 | 22.2 | 25.0 | 33.4 | 26.3 |
| Cold / cold weather | | | | | | | |
| Not at all | 47.5 | 61.3 | 55.4 | 41.7 | 53.6 | 48.1 | 42.1 |
| Slightly/somewhat worse | 44.3 | 30 | 36.5 | 52.8 | 42.9 | 44.5 | 47.4 |
| Much/very much worse | 8.2 | 8.8 | 8.1 | 5.6 | 3.6 | 7.5 | 10.6 |
| Certain types of clothes | | | | | | | |
| Not at all | 23 | 31.3 | 28.4 | 47.2 | 32.1 | 35.2 | 26.3 |
| Slightly/somewhat worse | 52.5 | 45 | 48.7 | 36.1 | 53.6 | 38.9 | 57.9 |
| Much/very much worse | 24.6 | 23.8 | 23 | 16.6 | 14.3 | 26.0 | 15.8 |
| Stress | | | | | | | |
| Not at all | 23 | 36.3 | 25.7 | 41.7 | 32.1 | 22.2 | 10.5 |
| Slightly/somewhat worse | 37.7 | 40.1 | 48.7 | 30.5 | 42.9 | 27.8 | 42.1 |
| Much/very much worse | 39.4 | 23.8 | 25.7 | 27.8 | 25.0 | 50.0 | 47.4 |

### Example 2a

Natural History Study Results Comparison with Prior Art Conditions

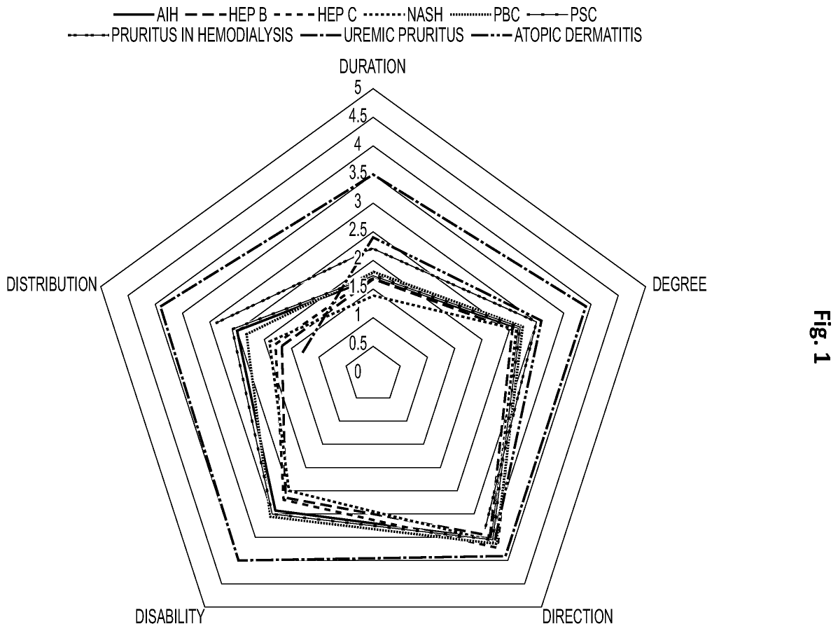

FIG. 1 shows a 5D-Itch Plot of Natural History Study Results for HepB, HepC, NASH (or MASH), PBC, PSC and AIH vs non-cholestatic prior art pruritus conditions (pruritus in hemodialysis, uremic pruritus and atopic dermatitis). FIG. 1a shows the same 5D-Itch Plot of Natural History Study Results, for NASH (or MASH) and PBC only. Participants in the natural history study of Example 1 completed the 5D-Itch measure using the 5D Itch Scale referenced hereinabove, that is, they completed the questionnaire provided in: 5D Itch Scale: Elman et al., “The 5-D itch scale: a new measure of pruritus.” Br J Dermatol. 2010 March; 162(3):587-93. The estimates presented are unadjusted, and patients and do not account for differences in liver disease duration or demographics between CLDs. Therefore, when comparing the five scores across domains for the different liver diseases, more emphasis is placed on degree and duration domains as these two domains capture the itch experience best in terms of severity and frequency, which are both key when assessing symptoms.

The values obtained from this evaluation are plotted against 5D-Itch measure values obtained from a targeted prior art search in which the 5D-Itch measure has been used for conditions where the itch is known not to be cholestatic in nature, namely pruritus in a hemodialysis patient population, a patient population with atopic dermatitis and a clinical trial patient population with uremic pruritus. All comparisons with these prior art studies are from non-interventional studies and all itch severities are included (mild, moderate and severe itch).

### Example 2a Results and Conclusions

As for Example 1, itch data from HepB, HepC and NASH (or MASH), as well as from PBC, PSC and AIH conditions from the natural history study are included in FIG. 1, since the itch associated with the latter group of conditions is known to be cholestatic in nature or have cholestatic features. The non-cholestatic itch experienced in the three non-cholestatic conditions from the prior art is notably different in terms of degree, duration and distribution from the cholestatic pruritus conditions. In contrast, itch in NASH (or MASH), HepB and HepC is similar to PSC, PBC and AIH in terms of degree and direction. Additionally, when comparing NASH (or MASH) and PBC (FIG. 1a), itch impact was comparable across many 5-D itch domains, including duration, degree, and direction.

This provides further supportive evidence relating to the similarities between itch in known cholestatic liver diseases, and itch in NASH (or MASH), AIH, HepB and HepC. Therefore, pruritus in NASH (or MASH), AIH, HepB and HepC may be amenable to treatment with an IBAT inhibitor.

### Example 3

### Qualitative Interviews to Characterize Patient Experience of Cholestatic Pruritus and Research Content Validity of Patient-Report Outcomes (PROs)

A qualitative study in patients with pruritus and chronic liver disease (CLD) which included patients with PSC, HepB, HepC, AIH, DILI NASH (or MASH) or NAFL (or MASLD) was set up as part of a program to select and validate patient reported outcomes (PROs) to assess itch and the impact that the itch has on patients with these conditions.

The principal study objective was as follows:
To characterize the patient experience pertaining to symptoms and impacts and identify the concepts of interest to patients with pruritus associated with CLD living in Canada, China, Germany, Japan, the United Kingdom (UK), or the United States (US).

Individuals with pruritus were invited, by a third-party vendor or a patient advocacy group, to participate in a 90-minute, one-on-one telephone or webcam interview conducted by a trained qualitative researcher. Each patient interview followed a standardized, semi-structured interview guide utilizing both CE (concept elicitation) and CD (cognitive debriefing) approaches to gather specific information related to the study objectives while also allowing for open-ended exploration of their pruritus as necessary. Table 4a outlines the sample in terms of CLDs and country distribution in the qualitative work.

TABLE 7
Participant Characteristics of CLD sample - liver conditions by country
N (%)

| Liver condition | US | UK | Canada | Germany | China | Japan | Total |
|---|---|---|---|---|---|---|---|
| Primary sclerosing | 4 (20) | 4 (25) | 4 (44) | 2 (13) | 3 (50) | 3 (38) | 20 (27) |
| cholangitis (PSC) | | | | | | | |
| Autoimmune | 2 (10) | 1 (6) | 1(11) | 1 (7) | 1 (17) | — | 6 (8) |
| hepatitis (AIH) | | | | | | | |
| Hepatitis B | 2 (10) | 1 (6) | — | 2 (13) | 1 (16.5) | 2 (25) | 8 (11) |
| Hepatitis C | 1 (5) | 1 (6) | 1(11) | 1 (7) | 1 (16.5) | 1 (12.5) | 6 (8) |
| Drug induced liver | 1 (5) | 1 (6) | 1(11) | 1 (7) | — | 1 (12.5) | 5 (7) |
| injury (DILI) | | | | | | | |
| Hepatitis B & C | — | 1 (6) | — | — | — | — | 1 (1) |
| AIH & PSC | — | 1 (6) | 1(11) | — | — | 1 (12) | 3 (4) |
| Non-alcoholic | 5 (25) | 4 (25) | 1(11) | 5 (33) | — | — | 15 (20) |
| steatohepatitis (or | | | | | | | |
| metabolic dysfunction- | | | | | | | |
| associated | | | | | | | |
| steatohepatitis) | | | | | | | |
| (NASH (or MASH)) | | | | | | | |
| Non-alcoholic fatty liver | 5 (25) | 2 (13) | — | 3 (20) | — | — | 10 (14) |
| (NAFL) | | | | | | | |
| (or Metabolic dysfunction | | | | | | | |
| associated steatotic liver | | | | | | | |
| disease (MASLD)) | | | | | | | |
| Total | 20 | 16 | 9 | 15 | 6 | 8 | 74 |

During the first part of the hybrid interviews, participants were asked open-ended questions about what it is like to have pruritus associated with their liver disease, and how it has affected their life. Participants, regardless of CLD or country of origin, were aligned in their descriptions of pruritus as an incessant, frustrating symptom that does not respond well to treatments and commonly leads to skin damage, including scarring, worse at night, disturbed sleep patterns, with no clear patterns of itching associated with season or weather, feelings of embarrassment and discomfort in social and professional situations, and a need to step back from recreational activities.

This study provides evidence that the patient experience of pruritus is similar among patients with different underlying liver conditions (PSC, AIH, Hep B, Hep C, DILI, NASH (or MASH), and NAFL) in Canada, China, Germany, Japan, the UK and the US. PSC is widely accepted to be a cholestatic liver disease; AIH has cholestatic features.

This evidence supports the qualitative findings from the Natural History Study of Example 1 in that patients with NASH (or MASH) describe their itch in similar terms as patients with known cholestatic diseases.

### Example 3a

Qualitative Interviews in Patients with Pruritus Secondary to Primary Biliary Cholangitis

A very similar separate study to Example 3 was also conducted during the course of the drug development program in the pruritus in PBC, a recognized cholestatic condition. The primary objective of this study was to confirm saturation of concepts of interest and cognitively test the English versions of the PRO measures selected for use in PBC. A total of 15 participants were recruited in the US and an additional 5 were English speaking Canadians. Study subjects were all adults between 18 and 80 years of age, with a self-reported formal diagnosis of PBC, and pruritus of at least moderate intensity during the past eight weeks.

This study assists in providing an approximate comparison of PBC itch with the itch in the CLDs of Example 3. The studies of Examples 3 and 3a were not conducted with the intention of comparison across studies, so data was not collected in a manner intended for direct comparison.

Below is a summary of the data and findings from the studies of Examples 3 and 3a relating to how patients described the severity, location and frequency of the itch.

### 1. Itch Severity

Table 8 illustrates itch severity across the whole CLD population in the qualitative study of Example 3. Mean worst itch scores over the past 8 weeks were either 7 or 8 depending on country (range 4-10). In the PBC qualitative study Example 3a, the mean worst itch score was 89 on a 0-100 NRS (Range 50-100), Table 9.

TABLE 8
Participant characteristics of CLD sample - itch severity

| | Mean (range) | | | | | | |
|---|---|---|---|---|---|---|---|
| | US | UK | Canada | Germany | China | Japan | Total |
| Age | 49 (29-72) | 47 (28-70) | 39 (21-66) | 52 (26-63) | 60 (52-76) | 54 (33-77) | 49 (21-77) |
| Severity of | 7 (4-10) | 6 (4-9) | 6 (4-8) | 6 (4-8) | 7 (5-9) | 6 (4-8) | 6 (4-10) |
| itching during | | | | | | | |
| the past 8 | | | | | | | |
| weeks (0-10) | | | | | | | |
| Worst itching | 8 (6-10) | 8 (6-9) | 8 (6-10) | 7 (4-10) | 8 (6-9) | 7 (5-10) | 8 (4-10) |
| during the | | | | | | | |
| past 8 weeks | | | | | | | |
| (0-10) | | | | | | | |
| Mildest itching | 4 (0-7) | 3 (0-8) | 3 (0-8) | 3 (0-5) | 3 (2-4) | 3 (1-6) | 3 (0-10) |
| during the | | | | | | | |
| past 8 weeks | | | | | | | |
| (0-10) | | | | | | | |

TABLE 9
Participant characteristics of PBC
sample (Example 3a)-itch severity

| | | Total |
|---|---|---|
| Itch Rating | | (N = 20) |
| Itch NRS: Rating of the severity | Mean (SD) | 89.3 (14.1) |
| of the itching at its worst | Median | 92.5 |
| (0 = No itching at all-100 = | Range | 50-100 |
| Worst itching you can imagine) | | |

### 2. Describe Itching

In the broad CLD population of Example 3, participants consistently described the itching sensation of pruritus as an incessant, deep, burning itch that is not relieved by scratching, and often made worse by it. It was also described as a crawling, tingling, or tickling sensation. A small number of participants (n=3) likened it to being bitten by fire ants. For the NASH (or MASH) population in particular (n=15), participants described pruritus as an incessant itch (n=13). It was also described as: a hot, burning sensation (n=4), feeling like your skin is crawling (n=3), a tingling sensation (n=2), unbearable (n=2), a deep itch (n=1), and a tickly sensation (n=1). NAFL (or MASLD) patients (n=10) described the itching associated with pruritus as incessant (n-=8), deep (i.e., a deep itch, under the skin; n=4), and unbearable (n=2). When comparing across the CLDs, there were no clear patterns to how participants in the CLD sample described their itch based on the liver condition they had. In the known cholestatic pruritus population of PBC in Example 3a, itch related signs and symptoms sub-domain most of the expressions referred to “itch” in general (by all 20 subjects), but other itch-related concepts were also used, including “Hives” (n=3) “Burning” (n3=), “Rash” (n=3), “tingling” (n=4), and “Dry patches” (n=1). Across both PBC and the other CLDs burning and tingling were descriptions used by participants to recount their itch.

### 3. Body Location

Participants with CLD in Example 3 experienced pruritus in different locations of their body, and all experienced it in multiple locations. The majority of the CLD participants experienced it in their torso (n=46), arms (n=43), and legs (n=41). Participants also reported experiencing it in their feet or toes (n=29), hands (n=24), head (n=17), face (n=7), and genital region (n=4). Most participants experienced the itching in the same place or places, however 15 participants reported that the location of the itch varied with no discernible pattern to predict where they might experience it next. Similar to describing the itching sensation, the location of itching was not predictable by liver condition.

In patients with PBC in Example 3a the most common location for itching as discovered through coding was legs, affecting 15 (75.0%) subjects, followed by arms (13 or 65.0%), torso—including back, abdomen, and chest (12 or 60.0%), and feet (10 or 50.0%). Table 10 shows the frequency of subject expressions of itch associated with specific location. The patterns of itch are similar between PBC and CLD with the legs, arms and torso being the most commonly effected areas.

TABLE 10
Spontaneous vs. Probed Symptom Expressions

| | Subjects N = 20 (100.0%) | | | |
|---|---|---|---|---|
| | | | Not | Not |
| | Spontaneous | Probed | Affected | Reported |
| SYMPTOM REPORTED | N (%) | N (%) | N (%) | N (%) |
| Itching-Related Signs | | | | |
| and Symptoms: | | | | |
| Hives | 1 (5%) | — | — | 19 (95%) |
| Itching on the Abdomen | 7 (35%) | 2 (10%) | — | 11 (55%) |
| Itching on the Arms | 11 (55%) | 2 (10%) | — | 7 (35%) |
| Itching on the Back | 9 (45%) | 1 (5%) | — | 10 (50%) |
| Itching on the Chest | 1 (5%) | — | — | 19 (95%) |
| Itching on the Face | 2 (10%) | 1 (5%) | — | 17 (85%) |
| Itching on the Feet | 3 (15%) | — | — | 17 (85%) |
| Itching on the Groin | 1 (5%) | — | — | 19 (95%) |
| Itching on the Hands | 3 (15%) | — | — | 17 (85%) |
| Itching on the Head | 6 (30%) | — | — | 14 (70%) |
| Itching on the Legs | 15 (75%) | — | — | 5 (25%) |
| Itching on the Side | 9 (45%) | 1 (5%) | — | 10 (50%) |
| Rash | 1 (5%) | — | — | 19 (95%) |

### Example 3 and 3a Conclusions

In summary, within the CLD samples of Example 3 the itch experienced across the different CLDs (PSC, AIH, Hep B, Hep C, DILI, NASH (or MASH), and NAFL) is observed to be similar. Notably, PSC is widely accepted to be a cholestatic liver disease and AIH has cholestatic features; certain other CLDs studied including HepB, HepC, NASH (or MASH) and NAFL (or MASLD) are not classically considered to be cholestatic.

In addition, the itch associated with the CLDs in Example 3 is not found to be qualitatively different to that experienced in PBC in Example 3a, and therefore there is reason to believe that the CLD itch, for example in AIH, HepB, HepC, NASH (or MASH) and NAFL (or MASLD), is qualitatively similar to PBC itch, which is widely accepted to be cholestatic in nature. Therefore, pruritus in CLDs such as AIH, HepB, HepC, NASH (or MASH) and NAFL (or MASLD) may be amenable to treatment with an IBAT inhibitor.

### Example 4

### Study Characterising the Burden and Clinical Trajectory of Pruritus in Primary Sclerosing Cholangitis (PSC) and Non-PBC/PSC Chronic Liver Disease

This ongoing observational cohort study contains both cross-sectional and longitudinal assessments in patients with PSC, NAFLD (or MASLD)/NASH (or MASH), chronic HBV, chronic HCV, AIH, and DILI. Cross-sectional data from patients with irritable bowel disease alone, as well as from healthy volunteers, will also be collected. Patient assessments are aligned with routine standard of care clinical visits (minimum of 2 measurements, for up to 48 weeks).

NAFLD (or MASLD) and NASH (or MASH) patients formed a single cohort.

The co-primary objectives of this study are:
a) determine the proportion of patients with NAFLD (or MASLD)/NASH (or MASH) who suffer with pruritus and
b) to quantify pruritus intensity, and how this varies as per the inherent clinical course of NAFLD (or MASLD)/NASH (or MASH). The NRS scale and 5-D Itch Score were used to assess prevalence and severity of pruritus. As noted above, the 5-D itch scale is a multidimensional questionnaire designed to measure the five dimensions: degree, duration, direction, disability and distribution and the NRS scale measures intensity or severity of itch, rather than frequency of itch.

The secondary objectives of this study are:
Correlate itch intensity with disease severity, serum liver biochemical parameters, and where possible, serum bile acid values
Assess the current unmet need of pruritus in NAFLD (or MASLD) with respect to currently available medical therapies.

### Example 4 Results

### Baseline Characteristics and Severity of Disease

This study was still recruiting patients when interim data was reported, having enrolled 91 patients with NAFLD (or MASLD)/NASH (or MASH) at that stage. Baseline characteristics of the enrolled subjects at the time of the interim report can be found in Table 11. Of the 91 patients included in the interim results, over half were classified as having severe disease (advanced fibrosis or cirrhosis), one third as having no or mild fibrosis, and 11 patients remained unclassified with respect to their disease extent.

At the conclusion of enrolment, a total of 200 patients had been recruited into the study (including the initial 91 patients from the interim analysis). Baseline characteristics of the 200 enrolled subjects is shown in Table 11a. The data shows similar trends to those seen at the interim stage in terms of severity of disease in the patients.

TABLE 11
Baseline characteristics of NAFLD (MASLD)/
NASH (MASH) cohort (N = 91)

| | Total | |
|---|---|---|
| | N = 91 | |
| Age mean (range) | 56.47 | (17 to 83) |
| Sex (male) n (%) | 49 | (53.85) |
| BMI, mean (SD) | 34.90 | (7.08) |
| Diabetes Mellitus, n (%) | 63 | (69.23) |
| HBA1c mmol/mol (n = 84) mean (SD) | 53.86 | (16.36) |
| Hypertension, n (%) | 41 | (45.05) |
| Disease Extentª, n (%) | | |
| No fibrosis/mild fibrosis | 30 | (32.97) |
| Advanced fibrosis | 19 | (20.88) |
| Cirrhosis | 31 | (34.07) |
| Unstaged | 11 | (12.09) |
| Cirrhosis^{b}, (n = 80) n (%) | | |
| Yes | 31 | (38.75) |
| No | 49 | (61.25) |
| TSBA (n = 80) Median/ | 6.50/14.71 | (4-16.5/21.53) |
| Mean (IQR/SD) | | |
| Liver stiffness by TE-Fibroscan, | 10.10 | (7.20-17.00) |
| kPa (n = 76) Median (IQR) | | |
| Liver Stiffness >8.0 kPa , n (%) | 53 | (69.74) |

^{a}Fibrosis scores were determined by liver biopsy. Where liver biopsy was not available, Fibroscan scores were used; <8.0 k Pa = no/mild fibrosis. Diagnosis of cirrhosis was made based on liver biopsy or clinical assessment.
^{b}N = 80 because 11 of 91 patients were not able to be definitively staged using the criteria above.

TABLE 11a
Baseline characteristics of NAFLD (MASLD)/
NASH (MASH) cohort (N = 200)

| | Total | |
|---|---|---|
| | N = 200 | |
| Age mean (range) | 56.86 | (17-84) |
| Sex (male) n (%) | 115 | (57.5%) |
| BMI, mean (SD) | 36.92 | (28.98) |
| Diabetes Mellitus, n (%) | 139 | (69.5%) |
| HBA1c mmol/mol | 52.18 | (16.10) |
| (n = 188) mean (SD) | | |
| Hypertension, n (%) | 101 | (50.5%) |
| Disease Extentª, n (%) | | |
| No fibrosis/mild fibrosis | 66 | (33.0%) |
| Advanced fibrosis | 47 | (23.5%) |
| Cirrhosis | 66 | (33.0%) |
| Unstaged | 21 | (10.5%) |
| Cirrhosis^{b}, (n = 179) n (%) | | |
| Yes | 66 | (36.9%) |
| No | 113 | (63.1%) |
| TSBA (n = 171) Median/ | 6.00/13.20 | (4-13/20.32) |
| Mean (IQR/SD) | | |
| Autotaxin ng/ml (n = 64) | 332.6/349.8 | (270.0-416.2/125.9) |
| Median/Mean (IQR/SD) | | |
| Liver stiffness by TE-Fibroscan, | 11.20 | (7.28-18.20) |
| kPa (n = 166) Median (IQR) | | |
| Liver Stiffness >8.0 kPa , n (%) | 118 | (71%) |

^{a}Fibrosis scores were determined by liver biopsy. Where liver biopsy was not available, Fibroscan scores were used; <8.0 k Pa = no/mild fibrosis. Diagnosis of cirrhosis was made based on liver biopsy or clinical assessment.
^{b}N = 179 because 21 of 200 patients were not able to be definitively staged using the criteria above.

### Severity of Itch

Patient measurement of itch was captured using both a 0-10 point NRS where itch severity was classified as no itch (NRS=0), mild itch (NRS 1-3), moderate itch (NRS 4-6), and severe itch (NRS≥7), and the 5-D itch score. The 5-D itch score and NRS scale were highly correlated (Rₛₚₑₐᵣₘₐₙ=0.921, p<0.0001, N=91 in the interim analysis stage and Rₛₚₑₐᵣₘₐₙ=0.8540, p<0.0001, N=200 in the full analysis).

Of the initial 91 patients, 62.64% (n=57) of patients experienced itch of any severity, with 47.25% (n=43) experiencing moderate to severe itch when asked about their worst itch in the past 2 weeks. When measuring the average itch, 61.54% (n=56) of patients experienced itch of any severity, with 40.66% (n=37) experiencing moderate to severe itch, Table 12. Pruritus of all levels of severity was observed in the different liver fibrosis stages and in both cirrhotic and non-cirrhotic NASH (or MASH)/NAFLD (or MASLD) patients.

Among the data from the full cohort of 200 enrolled patients, similar trends were observed, with 51.5% (n=103) of patients experienced itch of any severity, and 36.5% (n=73) experiencing moderate to severe itch when asked about their worst itch in the past 2 weeks. When measuring the average itch, 49.5% (n=99) of patients experienced itch of any severity, with 31.0% (n=62) experiencing moderate to severe itch, Table 12a.

TABLE 12
Pruritus prevalence and reported severity (N = 91)

| | Total | |
|---|---|---|
| | N = 91 | |
| NRS Scale Average Itch | | |
| No Itch^{a}, n (%) | 35 | (38.46) |
| Any Degree of Itch^{a}, n (%) | 56 | (61.54) |
| Mild^{b} | 19 | (33.93) |
| Moderate^{b} | 26 | (46.43) |
| Severe^{b} | 11 | (19.64) |
| Median (IQR)^{c} | 2 | (0-5) |
| NRS Scale Worst Itch | | |
| No Itch^{a}, n (%) | 34 | (37.36) |
| Any Degree of Itch^{a}, n (%) | 57 | (62.64) |
| Mild^{b} | 14 | (24.56) |
| Moderate^{b} | 19 | (33.33) |
| Severe^{b} | 24 | (42.11) |
| Median (IQR)^{c} | 3 | (0-7) |
| 5-D Itch Score | | |
| No Itch^{a}, n (%) | 32 | (35.16) |
| Presence of Itch^{a}, n (%) | 59 | (64.84) |
| Median 5-D Score (IQR) | 9 | (5-12) |

Note:
Average itch is NRS value based on average itch experienced within previous 2 weeks. Worst itch is NRS value based on worst itch experienced within previous 2 weeks
Note:
Mild = NRS 1-3, Moderate = NRS 4-6, Severe = NRS ≥7
^{a}Percentage calculated out of total
^{b}Percentage calculated among those with any degree of itch
^{c}Median (IQR) calculated among all patients

TABLE 12a
Pruritus prevalence and reported severity (N = 200)

| | Total | |
|---|---|---|
| | N = 200 | |
| NRS Scale Average Itch | | |
| No Itch^{a}, n (%) | 101 | (50.5%) |
| Any Degree of Itch^{a}, n (%) | 99 | (49.5%) |
| Mild^{b} | 37 | (37.4%) |
| Moderate^{b} | 43 | (43.4%) |
| Severe^{b} | 19 | (19.2%) |
| Median (IQR)^{c} | 0 | (0-4) |
| NRS Scale Worst Itch | | |
| No Itch^{a}, n (%) | 97 | (48.5%) |
| Any Degree of Itch^{a}, n (%) | 103 | (51.5%) |
| Mild^{b} | 30 | (29.1%) |
| Moderate^{b} | 37 | (35.9%) |
| Severe^{b} | 36 | (35.0%) |
| Median (IQR)^{c} | 1 | (0-6) |
| 5-D Itch Score | | |
| No Itch^{a}, n (%) | 86 | (43%) |
| Presence of Itch^{a}, n (%) | 114 | (57%) |
| Median 5-D Score (IQR)^{c} | 8.5 | (5-12) |

Note:
Average itch is NRS value based on average itch experienced within previous 2 weeks. Worst itch is NRS value based on worst itch experienced within previous 2 weeks
Note:
Mild = NRS 1-3, Moderate = NRS 4-6, Severe = NRS ≥7
^{a}Percentage calculated out of total
^{b}Percentage calculated among those with any degree of itch
^{c}Median (IQR) calculated among all patients

### Levels of TSBA

Of the initial 91 patients (n=80 with TSBA measures), non-overnight fasted mean TSBA levels were high in NAFLD (or MASLD)/NASH (or MASH) patients and increased with increasing itch severity; however, variability was high. TSBA levels were high in both cirrhotic and non-cirrhotic patients with moderate to severe pruritus (Table 13). In the non-cirrhotic subgroup, TSBA increased with increasing itch severity. Similar trends with TSBA levels and itch severity were observed in the full cohort (n=171 with TSBA measures), Table 13a.

TABLE 13
Mean TSBA (SD) (μmol/L) by liver-biopsy-confirmed
cirrhosis and itch severity (from N = 91 cohort)

| | No Itch, | Moderate/Severe |
|---|---|---|
| | (NRS = 0) | Itch, (NRS ≥ 4) |
| Cirrhotic | 27.20 (18.91) | 31.57 (38.19) |
| | (n = 10) | (n = 14) |
| Non-Cirrhotic | 5.33 (3.11) | 15.92 (20.53) |
| | (n = 18) | (n = 12) |

TABLE 13a
Mean TSBA (SD) (μmol/L) by liver-biopsy-confirmed
cirrhosis and itch severity (from N = 200 cohort)

| | No Itch, | Moderate/Severe |
|---|---|---|
| | (NRS = 0) | Itch, (NRS ≥ 4) |
| Cirrhotic | 23.24 (16.83) | 34.88 (39.39) |
| | (n = 25) | (n = 24) |
| Non-Cirrhotic | 6.42 (6.97) | 11.43 (15.61) |
| | (n = 53) | (n = 23) |

### Levels of Autotaxin (ATX)

Information on autotaxin was not available at the time of the interim analysis. A total of 64 patients from the full cohort of 200 enrolled patients had measured levels of autotaxin. Autotaxin levels were found to be higher among patients with moderate to severe pruritus, as compared to patients with no itch, and particularly among the cirrhotic patients (Table 14a); however, sample sizes are limited.

TABLE 14a
Mean autotaxin (SD) (ng/mL) by cirrhosis and itch severity-

| | No Itch, | Moderate/Severe |
|---|---|---|
| | (NRS = 0) | Itch, (NRS ≥ 4) |
| Cirrhotic | 371.6 (77.98) | 533.7 (136.4) |
| | (n = 7) | (n = 5) |
| Non-Cirrhotic | 331.3 (125.7) | 353.4 (111.2) |
| | (n = 30) | (n = 7) |

### Example 4 Conclusion

Approximately one in three NAFLD (or MASLD) patients experience moderate-severe pruritus. There are significantly higher serum bile acid and autotaxin levels in those with moderate-severe pruritus. In summary, increases of TSBA and autotaxin in NAFLD (or MASLD), particularly NASH (or MASH), which correlate with reported itch severity, are supported by the results of the study of Example 4.

### Example 5

Analysis of the Relationship Between Linerixibat Dose and Change in TSBA Over Time, and Change in Pruritus, in Subjects with PBC

The effect of linerixibat dose and regimen on daily TSBA profiles in Phase 1 and 2 studies of patients with PBC were used to develop a population dose-pharmacodynamic (k-PD) model to characterise the linerixibat dose-TSBA relationship. The following studies were included: study NCT01899703; GLIMMER (NCT02966834) and healthy volunteers (NCT02801981; NCT01607385; NCT01416324).

Simulations were performed to explore the effect of linerixibat dose and regimen on daily TSBA profiles. Individual Bayesian parameter estimates for subjects in GLIMMER, a Phase 2b study of linerixibat in patients with PBC and moderate to severe pruritus (NCT02966834), were used to derive the area under the TSBA concentration-curve (AUC₀₋₂₄). These post hoc estimates of AUC₀₋₂₄ were correlated with itch reported on a 0-10 numerical rating scale (NRS). In GLIMMER, 4 weeks single-blind placebo (baseline=Week 4) was followed by a randomised, double-blind 12-week treatment period with linerixibat or placebo (to Week 16), and a further 4 weeks single-blind placebo (to Week 20). Mean worst daily itch (MWDI) and monthly itch score (MIS) were calculated as described previously (Levy C, et al. Clin Gastroenterol Hepatol 2022; S1542-3565(22)01021-7). Itch responders were defined as having a ≥2 point improvement in itch score from baseline.

### Example 5 Results

The final population k-PD model successfully described the effect of linerixibat on TSBA in PBC. Linerixibat treatment resulted in a rapid dose-dependent decrease in TSBA AUC₀₋₂₄; the reduction in TSBA AUC₀₋₂₄ reached steady-state after approximately 10 days. Baseline TSBA concentrations did not correlate with change from baseline in MIS at Week 16 (r=−0.13, p=0.14). At Week 16, there was a moderate correlation between change in TSBA AUC₀₋₂₄ and change in MIS from baseline (r=0.27, p=0.002), which dissipated during the placebo washout period (Week 20; r=0.011, p=0.91). Change in TSBA AUC₀₋₂₄ strongly correlated with improvement in MWDI score from baseline over the 12-week treatment period (r=0.52, p<0.0001; FIG. 3). A ≥30% decrease in TSBA AUC₀₋₂₄ was approximately 64% associated with an itch response.

### Example 5 Conclusion

Linerixibat treatment leads to rapid and dose-dependent reductions in TSBA. Baseline TSBA levels do not correlate with on-treatment change in NRS itch score, suggesting they do not predict linerixibat response. Change in TSBA over the double-blind treatment period correlates significantly with, and can be predictive of, improvement in itch in patients with PBC.

### Example 6

Analysis of the Association Between Itch Response and Changes in ATX and FGF-19 Biomarker Levels Following Linerixibat Treatment in Patients with Cholestatic Pruritus Associated with PBC

The Phase 2b, placebo-controlled, dose-ranging GLIMMER study (NCT02966834) enrolled 147 adult patients with PBC and pruritus (Levy 2022 Clin Gastroenterol Hepatol. 2022, available online https://doi.org/10.1016/j.cgh.2022.10.032). Patients were randomized to receive linerixibat or placebo for 12 weeks (Weeks 4-16). Patients reported itch on a 0-10 numerical rating scale. As patient numbers were too low to analyse biomarkers in individual dose groups, the two twice daily (BID) dose groups, 40 and 90 mg BID which showed a greater response in pharmacodynamic markers compared to once daily dosing, were combined. Serum ATX and FGF-19 levels were compared post hoc in patients receiving linerixibat 40/90 mg BID or placebo, at baseline versus Week 16, in itch responders and non-responders. Responder analysis from individual dose groups is not shown. Itch responders were defined as patients with a monthly itch score improvement of ≥2 at Week 16 compared with baseline.

### Example 6 Results

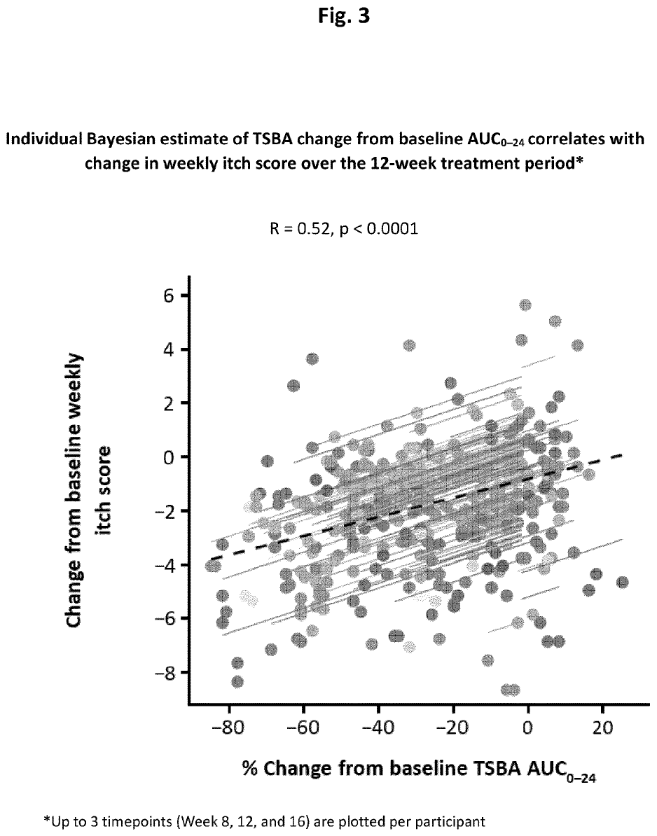

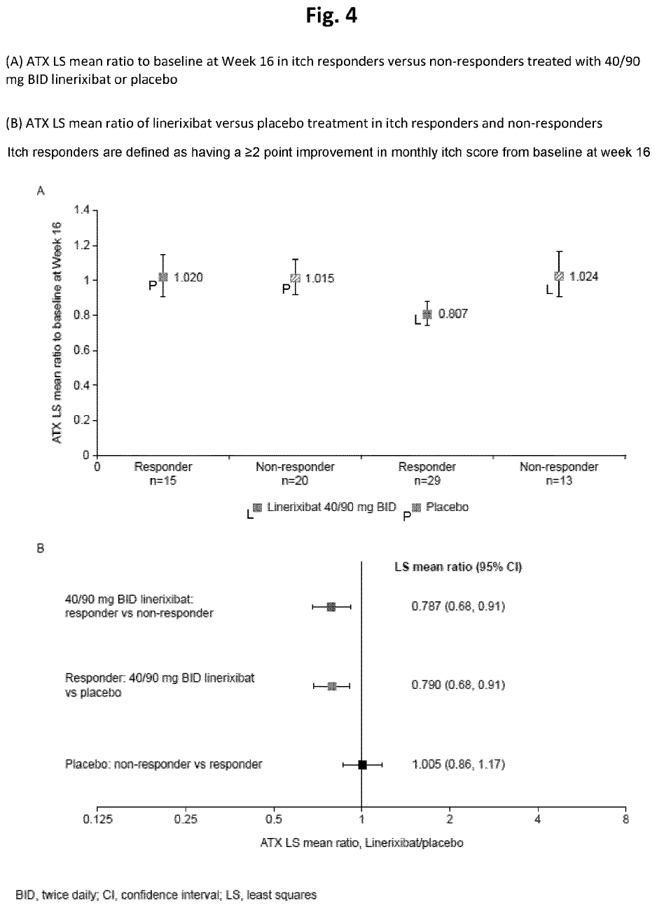

Patients treated with linerixibat 40/90 mg BID (n=45), or placebo (n=36) were included. Itch responders in the 40/90 mg BID group, but not in the placebo group, had greater reductions in serum ATX at Week 16 compared with baseline, FIG. 4a. In linerixibat-treated patients, ATX levels at Week 16 were reduced in itch responders compared to non-responders, and in comparison with placebo FIG. 4b. Similarly, FGF-19 reduction was associated with itch response in patients receiving linerixibat 40/90 mg BID compared with placebo.

### Example 6 Conclusions

Serum ATX and FGF-19 may function as biomarkers of itch response to linerixibat treatment. Reductions in these biomarkers are associated with clinical response in patients with PBC and pruritus.

### REFERENCES

Abdollahi, M. and M. Somi (2013). “Autoimmune hepatitis diagnosis and international criteria.” Journal of Gastroenterology and Hepatology 28: 167.
Bergasa, N. V., J. K. Mehlman and E. A. Jones (2000). “Pruritus and fatigue in primary biliary cirrhosis.” Baillieres Best Pract Res Clin Gastroenterol 14(4): 643-655.
Biernacka, A., D. Nizyński, M. Inglot and A. Reich (2018). “Assessment of pruritus in patients with viral hepatitis B and C.” Przeqlad Dermatologiczny 105(2): 264-272.
Boehlig, A., F. Gerhardt, D. Petroff, F. van Boemmel, T. Berg, V. Blank, T. Karlas and J. Wiegand (2022). “Prevalence of Pruritus and Association with Anxiety and Depression in Patients with Nonalcoholic Fatty Liver Disease.” Biomedicines 10(2).
Cacoub, P., T. Poynard, P. Ghillani, F. Charlotte, M. Olivi, J. C. Piette and P. Opolon (1999). “Extrahepatic manifestations of chronic hepatitis C. MULTIVIRC Group. Multidepartment Virus C.” Arthritis Rheum 42(10): 2204-2212.
European Association for the Study of the Liver. Electronic address, e. e. e. and L. European Association for the Study of the (2017). “EASL 2017 Clinical Practice Guidelines on the management of hepatitis B virus infection.” J Hepatol 67(2): 370-398.
Evon, D. M., H. H. S. Lin, M. Khalili, R. J. Fontana, C. Yim, A. S. Wahed, M. W. Fried, J. H. Hoofnagle, J. Niu, A. Javaid, B. Nasir, A. Susheela, I. Nasser, A. Donovan, N. Rusibamayila, C. Foley, A. C. Stahler, L. Stadheim, J. Lake, P. Lacher, K. Rushing, D. DeMarco Shaw, L. Kessels, M. K. Klebert, S. Noureldin, D. La, L. Liu, D. Kaznowski, J. Chen, F. Huang, D. Vladutu, O. Cerocchi, D. Rowan, S. Bass, B. Lilly, S. French, V. Peacock, D. Geffen, M. Peters, A. Shobe, R. Davis, R. Kuras, C. Ayala, I. Lau, V. Podolskaya, A. von Bakonyi, N. DeVole, B. McKenna, K. Choi, K. Oberhelman, S. Kaza, I. Moran, L. Huddleston, R. Wong, A. S. Barritt, T. Marsh, V. Metheny, D. Cardona, P. G. Smith, C. Hofmann, A. Wolfstone, J. Mooney, L. Cardona-Gonzalez, N. Fryzek, E. Rivera, N. Morris, V. Haynes-Williams, A. Huang, C. Nadal, J. Norman-Wheeler, A. H. Sherker, R. J. Torrance, S. R. Hall, M. E. Valiga, K. Torrey, D. Levine, J. Keith, M. Betts, L. J. Montaner, F. Averbach, T. Haller, R. Hardison, S. Kelley, C. Lalama, S. Lawlor, M. Lombardero, A. Pelesko, D. Stoliker, M. Weiner, E. Zadorozny and Q. Zhao (2020). “Patient-reported outcomes in a large North American cohort living with chronic hepatitis B virus: a cross-sectional analysis.” Alimentary Pharmacology and Therapeutics 51(4): 457-468.
Fattovich, G. (2003). “Natural history of hepatitis B.” J Hepatol 39 Suppl 1: S50-58.
Fiorucci, S., M. Biagioli and E. Distrutti (2018). “Future trends in the treatment of non-alcoholic steatohepatitis.” Pharmacological Research 134: 289-298.
Foster, C., J. Baki, S. Nikirk, S. Williams, N. D. Parikh and E. B. Tapper (2020). “Comprehensive Health-State Utilities in Contemporary Patients With Cirrhosis.” Hepatology Communications 4(6): 852-858.
Fujino, H., M. Tanaka, M. Imamura, K. Morio, A. Ono, T. Nakahara, E. Murakami, T. Kawaoka, S. Takahashi, D. Miki, M. Tsuge, A. Hiramatsu, H. Aikata, C. N. Hayes and K. Chayama (2019). “Pruritus in patients with chronic liver disease and serum autotaxin levels in patients with primary biliary cholangitis.” BMC Gastroenterol 19(1): 169.
Gordon, V., R. Adhikary, V. Appleby, D. Das, J. Day, T. Delahooke, S. Dixon, D. Elphick, C. Hardie, B. Hoeroldt, P. Hooper, J. Hutchinson, R. Jones, F. Khan, G. P. Aithal, J. McGonigle, A. Nelson, A. Nkhoma, S. Pelitari, M. Prince, A. Prosser, V. Sathanarayana, S. Savva, N. Shah, S. Saksena, S. Thayalasekaran, D. Vani, A. Yeoman and D. Gleeson (2018). “Diagnosis, presentation and initial severity of Autoimmune Hepatitis (AIH) in patients attending 28 hospitals in the UK.” Liver International 38(9): 1686-1695.
Gunsar, F., B. Gursel, F. Yilmaz, I. Turan, F. Tekin, Z. Karasu, G. Ersoz and U. S. Akarca (2016). “Clinical presentations of autoimmune hepatitis.” Hepatology International 10(1): S180.
Hegade, V. S., R. Bolier, R. P. J. Oude Elferink, U. Beuers, S. Kendrick and D. E. J. Jones (2016). “A systematic approach to the management of cholestatic pruritus in primary biliary cirrhosis.” Frontline Gastroenterology 7(3): 158-166.
Hegade, V. S., S. F. Kendrick, R. L. Dobbins, S. R. Miller, D. Thompson, D. Richards, J. Storey, G. E. Dukes, M. Corrigan, R. P. J. Oude Elferink, U. Beuers, G. M. Hirschfield and D. E. Jones (2017). “Effect of ileal bile acid transporter inhibitor GSK2330672 on pruritus in primary biliary cholangitis: a double-blind, randomised, placebo-controlled, crossover, phase 2a study.” Lancet 389(10074): 1114-1123.
Hegade, V. S., G. F. Mells, H. Fisher, S. Kendrick, J. DiBello, K. Gilchrist, G. J. Alexander, G. M. Hirschfield, R. N. Sandford, D. E. J. Jones and U.-P. Consortium (2019). “Pruritus Is Common and Undertreated in Patients With Primary Biliary Cholangitis in the United Kingdom.” Clin Gastroenterol Hepatol 17(7): 1379-1387 e1373.
Hönig, S., B. Herder, A. Kautz, C. Trautwein and A. Kremer (2018). “Pruritus strongly reduces quality of life in PBC patients-real life data from a large national survey.” Journal of Hepatology 68: S216.
Jin, X. Y. and T. M. Khan (2016). “Quality of life among patients suffering from cholestatic liver disease-induced pruritus: A systematic review.” J Formos Med Assoc 115(9): 689-702.
Kido-Nakahara, M., T. Nakahara, N. Furusyo, S. Shimoda, K. Kotoh, M. Kato, J. Hayashi, T. Koyanagi and M. Furue (2019). “Pruritus in chronic liver disease: A questionnaire survey on 216 patients.” Acta Dermato-Venereologica 99(2): 220-221.
Koay, L. B., C. Y. Lin, S. L. Tsai, C. Lee, C. N. Lin, M. J. Sheu, H. T. Kuo and C. S. Sun (2006). “Type 1 autoimmune hepatitis in Taiwan: Diagnosis using the revised criteria of the International Autoimmune Hepatitis Group.” Digestive Diseases and Sciences 51(11): 1978-1984.
Kremer, A. E., U. Beuers, R. P. J. Oude-Elferink and T. Pusl (2008). “Pathogenesis and treatment of pruritus in cholestasis.” Drugs 68(15): 2163-2182.
Kremer, A. E., R. P. J. Oude Elferink and U. Beuers (2011). “Pathophysiology and current management of pruritus in liver disease.” Clinics and Research in Hepatology and Gastroenterology 35(2): 89-97.
Liberal, R., E. L. Krawitt, J. M. Vierling, M. P. Manns, G. Mieli-Vergani and D. Vergani (2016). “Cutting edge issues in autoimmune hepatitis.” J Autoimmun 75: 6-19.
Lindor, K. D., C. L. Bowlus, J. Boyer, C. Levy and M. Mayo (2019). “Primary Biliary Cholangitis: 2018 Practice Guidance from the American Association for the Study of Liver Diseases.” Hepatology 69(1): 394-419.
Manns, M. P., A. W. Lohse and D. Vergani (2015). “Autoimmune hepatitis—Update 2015.” J Hepatol 62(1 Suppl): S100-111.
Mieli-Vergani, G., D. Vergani, A. J. Czaja, M. P. Manns, E. L. Krawitt, J. M. Vierling, A. W. Lohse and A. J. Montano-Loza (2018). “Autoimmune hepatitis.” Nature Reviews Disease Primers 4(1): 18017.
Montagnese, S., L. M. Nsemi, N. Cazzagon, S. Facchini, L. Costa, N. V. Bergasa, P. Amodio and A. Floreani (2013). “Sleep-Wake profiles in patients with primary biliary cirrhosis.” Liver International 33(2): 203-209.
Muratori, P., A. Fabbri, C. Lalanne, M. Lenzi and L. Muratori (2015). “Autoimmune liver disease and concomitant extrahepatic autoimmune disease.” Eur J Gastroenterol Hepatol 27(10): 1175-1179.
NICE (2013). Hepatitis B (chronic): diagnosis and management.
Oeda, S., H. Takahashi, H. Yoshida, Y. Ogawa, K. Imajo, M. Yoneda, Y. Koshiyama, M. Ono, H. Hyogo, T. Kawaguchi, H. Fujii, K. Nishino, Y. Sumida, S. Tanaka, M. Kawanaka, T. Torimura, T. Saibara, A. Kawaguchi, A. Nakajima and Y. Eguchi (2018). “Prevalence of pruritus in patients with chronic liver disease: A multicenter study.” Hepatology Research 48(3): E252-E262.
Perryman, S., G. Sreepati, N. Chalasani and C. Lammert (2014). “Fatigue and itch are associated with less biochemical response in treated autoimmune hepatitis.” American Journal of Gastroenterology 109: S137-S138.
Powell, E. E., V. W. Wong and M. Rinella (2021). “Non-alcoholic fatty liver disease.” Lancet 397(10290): 2212-2224.
Rishe, E., A. Azarm and N. V. Bergasa (2008). “Itch in primary biliary cirrhosis: A patients' perspective.” Acta Dermato-Venereologica 88(1): 34-37.
Suzuki, K., M. Tamano, Y. Katayama, T. Kuniyoshi, K. Kagawa, H. Takada and K. Suzuki (2014). “Study of pruritus in chronic hepatitis C patients.” World Journal of Gastroenterology 20(47): 17877-17882.
Tang, L. S. Y., E. Covert, E. Wilson and S. Kottilil (2018). “Chronic Hepatitis B infection a review.” JAMA—Journal of the American Medical Association 319(17): 1802-1813.
Terrault, N. A., A. S. F. Lok, B. J. McMahon, K. M. Chang, J. P. Hwang, M. M. Jonas, R. S. Brown, Jr., N. H. Bzowej and J. B. Wong (2018). “Update on Prevention, Diagnosis, and Treatment of Chronic Hepatitis B: AASLD 2018 Hepatitis B Guidance.” Clin Liver Dis (Hoboken) 12(1): 33-34.
Vander Does, A., C. Levy and G. Yosipovitch (2022). “Cholestatic Itch: Our Current Understanding of Pathophysiology and Treatments.” Am J Clin Dermatol.
Wedemeyer, H., G. J. Dore and J. W. Ward (2015). “Estimates on HCV disease burden worldwide—filling the gaps.” J Viral Hepat 22 Suppl 1: 1-5.
Werner, M., S. Wallerstedt, S. Lindgren, S. Almer, E. Björnsson, A. Bergquist, H. Prytz, H. Sandberg-Gertzén, R. Hultcrantz, P. Sangfelt, O. Weiland, B. Ohlsson and A. Danielsson (2010). “Characteristics and long-term outcome of patients with autoimmune hepatitis related to the initial treatment response.” Scandinavian Journal of Gastroenterology 45(4): 457-467.
Whalley, D., J. Twiss, L. Doward, M. M. Balp, C. Brass, A. Tietz, J. Loeffler, P. Lopez, E. J. Lawitz and A. J. Sanyal (2020). “A novel patient-reported outcome measure indicates low burden of treatment among patients with nonalcoholic steatohepatitis: Interim results from a phase 2 trial of tropifexor.” Hepatology 72(1 SUPPL): 1005A.
WHO, W. H. O. (2016). Guidelines for the screening, care and treatment of persons with chronic hepatitis C infection.
WHO, W. H. O. (2019). “Hepatitis C.” Retrieved 7th May 2020, 2020, from https://www.who.int/news-room/fact-sheets/detail/hepatitis-c.
Yerragorla, P., D. Nekkalapudi and N. V. Bergasa (2017). “Characterization of patients with primary biliary cholangitis (PBC) and autoimmune hepatitis (AIH) attended in a community hospital in East Harlem, New York.” Hepatology 66: 200A.
Younossi, Z., Q. Anstee, V. W. S. Wong, M. Trauner, M. Camargo, Z. Goodman, L. Henry, M. Stepanova, M. Romero-Gomez, R. Myers and E. Lawitz (2020). “High prevalence of fatigue and pruritus in patients with advanced fibrosis due to nonalcoholic steatohepatitis (Nash): The impact on patient-reported outcomes (Pros).” Diabetes 69.
Younossi, Z. M., M. L. Kiwi, N. Boparai, L. L. Price and G. Guyatt (2000). “Cholestatic liver diseases and health-related quality of life.” Am J Gastroenterol 95(2): 497-502.
Zaltron, S., A. Spinetti, L. Biasi, C. Baiguera and F. Castelli (2012). “Chronic HCV infection: epidemiological and clinical relevance.” BMC Infect Dis 12 Suppl 2: S2.
Mayo M J, et al. Impact of Pruritus on Quality of Life and Current Treatment Patterns in Patients with Primary Biliary Cholangitis. Dig Dis Sci. 2023 March; 68(3):995-1005.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. A method of treatment of pruritus in NAFLD or MASLD in a human in need thereof, comprising administering to said human a therapeutically effective amount of an IBAT inhibitor.

15. A method of treatment of cholestatic pruritus in NAFLD or MASLD in a human in need thereof, comprising administering to said human a therapeutically effective amount of an IBAT inhibitor.

16. A method of treatment according to claim 14 wherein the IBAT inhibitor is selected from:
maralixibat, volixibat, odevixibat, elobixibat, linerixibat and Albireo A-3907;
or a pharmaceutically acceptable salt or crystalline form thereof.

17. A method of treatment according to claim 14, wherein the IBAT inhibitor is linerixibat, or a pharmaceutically acceptable salt or crystalline form thereof.

18. A method of treatment of according to claim 14, wherein the NAFLD or MASLD is NASH or MASH.

19. A method of treatment of according to claim 14, wherein the NAFLD is NAFL.

20. A method of treatment of according to claim 14, wherein the NAFLD or MASLD is noncirrhotic NASH or MASH.

21. A method of treatment of according to claim 14, wherein the NAFLD or MASLD is NASH or MASH with cirrhosis.

22. A method of treatment of according to claim 14, wherein the IBAT inhibitor is present as the free acid.

23. A method of treatment of according to claim 14, wherein the IBAT inhibitor is present in crystalline form.

24. The method of treatment according to claim 27, wherein moderate to severe pruritus is measured as ≥4 on an NRS scale.

25. The method of treatment according to claim 27, wherein a patient with cirrhosis has a TSBA level of about 34.

26. The method of treatment according to claim 27, wherein a patient without cirrhosis has a TSBA level of about 11.

27. A method of treatment according to claim 14, wherein the pruritus is moderate to severe.

28. A method of treatment according to claim 15 wherein the IBAT inhibitor is selected from:
maralixibat, volixibat, odevixibat, elobixibat, linerixibat and Albireo A-3907;
or a pharmaceutically acceptable salt or crystalline form thereof.

29. A method of treatment according to claim 15, wherein the IBAT inhibitor is linerixibat, or a pharmaceutically acceptable salt or crystalline form thereof.

30. A method of treatment of according to claim 15, wherein the NAFLD or MASLD is NASH or MASH.

31. A method of treatment of according to claim 15, wherein the NAFLD is NAFL.

32. A method of treatment of according to claim 15, wherein the NAFLD or MASLD is noncirrhotic NASH or MASH.

33. A method of treatment of according to claim 15, wherein the NAFLD or MASLD is NASH or MASH with cirrhosis.

34. A method of treatment of according to claim 15, wherein the IBAT inhibitor is present as the free acid.
